(19) United States
(12) Patent Application Publication
(10) Pub. No.: US 2016/0186263 A1
(43) Pub. Date: Jun. 30, 2016

(54) USING PLEXIN-A4 AS A BIOMARKER AND THERAPEUTIC TARGET FOR ALZHEIMER'S DISEASE

(71) Applicant: TRUSTEES OF BOSTON UNIVERSITY, Boston, MA (US)

(72) Inventors: Lindsay A. FARRER, Wayland, MA (US); Gyungah JUN, Malden, MA (US)

(21) Appl. No.: 14/889,565

(22) PCT Filed: May 9, 2014
(86) PCT No.: PCT/US2014/037479

§ 371 Date: Nov. 6, 2015

Related U.S. Application Data
(60) Provisional application No. 61/821,397, filed on May 9, 2013.

(51) Int. Cl. C12Q 1/68; C12N 15/113

(52) U.S. Cl. CPC C12Q 1/6883; C12N 15/1138; C12Q 2600/158; C12Q 2600/156; C12Q 2600/118; C12N 2320/34

(57) ABSTRACT

The present disclosure provides methods, assays and systems for detecting an increased risk for Alzheimer's disease (AD) in a subject by identifying at least one nucleic acid polymorphism described herein in a biological sample from the subject. Levels of the genes associated with the nucleic acid polymorphism described herein are also determined for detection of higher risk for AD. Disclosure further provides methods for treating AD by administering to a subject in need thereof a TS1 PLXNA4 inhibitory agent.

### RELATED APPLICATIONS

This application claims benefit under 35 U.S.C. §119(e) of the U.S. Provisional Application No. 61/821,397, filed May 9, 2013, the content of which is incorporated herein by reference in its entirety.

### GOVERNMENT SUPPORT

This invention was made with Government Support under Contract No.: AG25259 awarded by the National Institutes of Health. The Government has certain rights in the invention.

### TECHNICAL FIELD

The present invention relates generally to assays, methods, and systems for diagnosing Alzheimer's disease (AD). The invention further relates to methods and compositions for treatment of AD.

### BACKGROUND

Alzheimer's disease is a leading cause of dementia in the elderly, affecting 5-10% of the population over the age of 65 years (A Guide to Understanding Alzheimer's Disease and Related Disorders, Jorm, ed., New York University Press, New York, 1987). In Alzheimer's disease, the parts of the brain essential for cognitive processes such as memory, attention, language, and reasoning degenerate, robbing victims of much that makes us human, including independence. In some forms of Alzheimer's disease, onset can first be seen in middle age, but more commonly, symptoms appear from the 65 and onwards. Alzheimer's disease today affects 4-5 million Americans, with slightly more than half of these people receiving care at home, while the others are in many different health care institutions. The prevalence of Alzheimer's disease and other dementias doubles every 5 years beyond the age of 65, and recent studies indicate that nearly 50% of all people age 85 and older have symptoms of Alzheimer's disease (1999 Progress Report on Alzheimer's Disease, National Institute on Aging/National Institute of Health). 13% (33 million people) of the total population of the United States are age 65 and older, and this percentage will climb to 20% by the year 2025 (1999 Progress Report on Alzheimer's Disease).

Alzheimer's disease also puts a heavy economic burden on society. A recent study estimated that the cost of caring for one Alzheimer's disease patient with severe cognitive impairments at home or in a nursing home, is more than $47,000 per year (A Guide to Understanding Alzheimer's Disease and Related Disorders). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (1999 Progress Report on Alzheimer's disease).

### SUMMARY

Provided herein are several applications involving methods and assays comprising PLXNA4. In some aspects, provided herein are methods of treating Alzheimer disease (AD) by inhibiting phosphorylation of tau inhibitor via inhibition of PLXNA4 or one of its ligands including, but not limited to, SEMA3. In some aspects, PLXNA4 can be used as a biomarker by measuring the expression levels of the full-length and shorter PLXNA4 isoforms in serum or by genotyping PLXNA4 single nucleotide polymorphisms (SNPs) that are genetically associated with AD including, but not limited to, rs277470, rs277472, rs277473, rs277474, rs277476, rs277477, rs277478, rs277479, rs277480, rs277481, rs277483, rs277484, rs10234979, rs9641933, rs10225863, rs7799929, rs9656410, rs11764790, rs4731860, rs11763817, rs13231950, rs11773243, rs17166338, rs11773724, rs17818325, rs10236235, rs965800, rs10244929, rs4481530, rs1593222, rs1424590, rs1364505, rs6959579, and rs11761937. As such, a PLXNA4 biomarker can be used for predictive testing for future development of AD and for classifying subjects enrolled in clinical trials as having high or low risk of developing AD.

As described herein, association of Alzheimer's disease with 341,492 genotyped single nucleotide polymorphisms (SNPs) was evaluated in the Framingham Heart Study (FHS) cohort comprising 61 incident cases and 2,530 cognitively normal individuals using an approach that accounts for family structure. Top-ranked SNPs were genotyped in a replication cohort containing 1,840 cases and 1,969 unaffected individuals in the National Institute on Aging Late Onset Alzheimer Disease (NIA-LOAD) Study.

As described herein, genome-wide significant associations were identified in the FHS with moderately rare SNPs in ITIH3 (rs9311482, p=4.6×10⁻⁹), PLXNA4 (rs277484) p=9.0×10⁻¹° and MY018B (rs13057714, p=8.9×10⁻⁹). As demonstrated herein, ten PLXNA4 SNPs were significant in NIA-LOAD after multiple testing correction in a propensity score model which conditioned on parental affection status and onset age (rs11761937, p=5.8×10⁻⁶). Transfection of SH-SY5Y cells or primary rat neurons with the full-length PLXNA4 isoform (TS1) increased tau phosphorylation and formation of neurofibrillary tangles when stimulated by semaphorin-3A, whereas the opposite effect was observed when transfected with shorter isoforms (TS2 and TS3). Transfection of any isoform into HEK293 cells did not affect APP processing or Aβ production. Late-stage AD cases (n=9) compared to controls (n=5) had 1.9-fold increased expression of TS1 in cortical brain tissue (P=1.6×10⁻⁴). Risk alleles from several AD-associated SNPs were significantly correlated with elevated expression of TS1 and TS3 in serum from 116 population controls.

The data reported herein shows that PLXNA4-mediated tau phosphorylation is an independent upstream event leading to AD-related tangle formation in neurons, and that this process is modulated by the level of the TS1 and TS3 isoforms. The results reported herein also show that reduced expression of PLXNA4, for example the TS1 isoform in particular, in brain is crucial to healthy neurons. The results described herein further show that PLXNA4 has a role in AD pathogenesis through isoform-specific effects on tau phosphorylation. Thus, without wishing to be bound by a theory, PLXNA4 or its binding partners can be used as novel drug targets for AD, as well as markers in assays for classifying and identifying subjects at risk for Alzheimer's disease, such as serum-based assays.

Accordingly, provided herein, in some aspects are assays using PLXNA4 as a biomarker in serum, assays and methods using PLXNA4 variants for predictive testing or stratification of subjects in clinical trials and for treatment purposes, and PLXNA4 variants, and their binding partners, as targets in methods for treatment of Alzheimer's disease.

In one aspect provided herein are assays and methods for determining an increased risk for developing AD in a subject. In one aspect, the assay and method comprise (a) transforming a biological sample from the subject into at least one detectable target loci for a nucleic acid polymorphism, wherein the target locus is selected from: SNP rs277472, SNP rs10236235, and SNP rs11761937; and (b) detecting presence or absence of at least one (e.g., one, two, three or more) AD risk associated SNPs from the at least one detectable target loci. In some embodiments, AD risk associated SNPs include A/A or A/C SNP rs277472, T/T or T/C SNP rs10236235, and C/C or C/A SNP rs11761937.

Another aspect of the assays and methods for determining an increased risk for developing AD in a subject include measuring the amount of at least one gene associated with the AD risk associated SNPs described herein in a biological sample from the subject, and then comparing the measured amount of the gene to a reference amount. In some embodiments, at least the amount of PLXNA4 gene expression products (e.g., nucleic acid or protein) associated with SNP rs277472, SNP rs10236235, or SNP rs11761937 is measured in a biological sample of a subject and compared to a reference level. In some embodiments, expression of full length isoform 1 (TS1) or shorter isoform 3 (TS3) of PLXNA4 gene expression product is measured. If the amount of the PLXNA4 gene expression products is higher than that of the reference amount, the subject is at increased risk for developing AD. The amount of the PLXNA4 gene expression products can be higher by at least about 10%, at least about 20%, at least about 30%, at least about 40%, at least about 50%, at least about 60%, at least about 70%, at least about 80%, at least about 90%, or at least about 95%, about 98%, about 99% or 100%, or higher than the reference level.

Without limitations the reference amount can be that measured in a normal healthy subject with no genetic susceptibility for AD. For example, a normal healthy subject that is not a carrier of any of the AD risk associated alleles described herein or is not diagnosed with any forms of AD such as early-onset autosomal-dominant AD, or any neurodegenerative disorders. The reference amount can be from a control sample, a pooled sample of control individuals or a numeric value or range of values based on the same.

In some embodiments, the invention provides methods or assays for determining if an individual is in need of AD treatment or prevention, comprising the steps of determining if the subject carries any of the SNPs selected from the group consisting of: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof. If the subject carries any of the SNPs, then the subject can further be administered a treatment or prevention intervention to treat AD symptoms or inhibit development of AD symptoms. These treatment of prevention interventions include, but are not limited to life style advise, including e.g., prescribing an aerobic exercise regime, dietary advise, including increase in intake of omega-3 fatty acids or reduction of sugar or cholesterol rich food intake to lower cholesterol, and administering pharmaceutical agents effective in prevention or treatment of AD. In some embodiments, the treatment includes administering a therapeutically effective amount of a TS1 PLXNA4 inhibitory agent.

A further aspect of the invention provides a computer implemented system for determining presence or absence of alleles associated with an increased risk of a subject for developing late onset Alzheimer's disease (AD). The system comprises (a) a determination module configured to identify and detect at least one single nucleotide polymorphism (SNP) in a biological sample of a subject, wherein the SNP is selected from: (i) (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof; (b) a storage module configured to store output data from the determination module; (c) a computing module adapted to identify whether at least one AD risk associated SNP is present or absent in the output data stored on the storage module; and (d) a display module for displaying if any of the AD risk associated SNP was identified or not. In one embodiment, the display module can display the detected alleles.

Yet another aspect of the invention relates to a pharmaceutical composition and methods for treating AD in a subject. The method comprises administering to the subject a pharmaceutically acceptable composition comprising a TS1 PLXNA4 inhibitory agent. In some embodiments, the method further comprises diagnosing the individual as having or at risk of AD prior to administering the agent. The diagnosing can be performed, e.g, using the method of determining the level of PLXNA4 (e.g., isoform TS1 or TS3) or by detecting the presence or absence of any one or more of the AD associated SNPs disclosed herein.

A still yet another aspect of the invention relates to a method for determining if a subject is in need of treatment or prevention for AD. The method comprises the steps of: (a) transforming at least one (e.g., one, two, or three) nucleic acid polymorphism in a locus in a biological sample from the subject into at least one detectable target, wherein the locus is selected from SNP rs277472, SNP rs10236235, or SNP rs11761937; and (b) detecting presence or absence of at least one AD risk associated SNP from the at least one detectable target, wherein detection of the presence of at least one AD risk associated allele is indicative of the subject in need for treatment or prevention for AD. In some embodiments, the method further comprises administering a treatment or preventive intervention to the subject, if presence of at least one AD risk-associated SNP is detected.

In another aspect, the disclosure provides a assay for identifying a subject having or at risk for Alzheimer's disease. The method comprising measuring or quantifying the expression level or amount of one or both of TS1 and TS3 PLXNA4 transcripts (e.g., mRNA) in a biological sample obtained from the subject and identifying the subject as having or at risk for Alzheimer's disease if the expression level or amount of one or both of TS1 and TS3 PLXNA4 transcript is increased relative to a reference value.

### BRIEF DESCRIPTION OF THE DRAWINGS

FIGS. 1A-1F show genetic findings in the PLXNA4 region. Regional association plots of genotyped and imputed SNPs from the FHS (1A) and NIA-LOAD (1B) datasets. Most significant SNPs in the FHS (rs277472) and NIA-LOAD (rs11761937) datasets are indicated by purple diamonds. P-values are expressed as −log 10(P) (y-axis) for every tested SNP ordered by chromosomal location (x-axis). Estimates of linkage disequilibrium (r²) of SNPs in this region with the top SNP computed using 1000 Genomes (hg19/November 2010EUR) are shown as circles for r²≧0.8, circles for 0.5≦r²<0.8, circles for 0.2≦r²<0.5, and circles for r²<0.2. (1C) Genomic structure was determined using the NCBI database (Build 37.1). Three validated transcripts (TS1, TS2, and TS3) are shown. Top association signals are highlighted in pink for the FHS dataset and in yellow for the NIA-LOAD dataset. The gene structure and reading frame are indicated with a pink arrow. Exons are denoted with vertical bars on the arrow. Predicted impact of rs277472 (1D) and rs11761937/rs10236235 (1E) on intronic splicing enhancer elements is shown. Protective (left-side nucleotide) and risk (right-side nucleotide) alleles are shown with predicted motifs inside the rectangles. (1F) Diagrams of functional domains encoded by amino acids from the full-length (TS1) and the shorter (TS2 and TS3) transcripts. SEMA: sema_plexinA1 interacting module; PSI: plexin repeat; IPT: three repeats of the binding domains of plexins and cell surface receptors; IPT_PCSR: binding domain of plexins and cell surface receptors (PCSR) and related proteins; TM: transmembrane region; CYTO: cytoplasmic domain. Sequences show in FIG. 1D are (i) GGTCCTCGCCTCC (SEQ ID NO: 2) and (ii) GGTCCTAGCCTCC (SEQ ID NO: 3); in FIG. 1E are (i) GTTTGCCGTGTCG (SEQ ID NO: 4); (i) GTTTGCTGTGTCG (SEQ ID NO: 5); (iii) TCCCAAACTCCTG (SEQ ID NO: 6); and (iv) TCCCAACCTCCTG (SEQ ID NO: 7).

FIGS. 2A-2C show PLXNA4 isoforms in tau phosphorylation. (2A) SH-SY5Y P301L cells were transfected with the full-length (TS1) or one of the shorter isoforms (TS2 and TS3) of PLXNA4-Myc or empty vectors (pcDNA3.1) with or without 3 nM SEMA3A stimulation for lhr. Whole cell lysates were blotted with AT8, total tau, actin and Myc. Results for the TS2 and TS3 isoforms were similar but only those for TS3 are shown. (2B) 6×His-tagged SEMA3A-Fc was precipitated from media by Protein A/G agarose, and the precipitates were immunoblotted with antibodies to Myc (detecting PLXNA4 isoforms) and 6×His (detecting SEMA3A-FC). (2C) Rat hippocampal neurons were transfected with the full-length or short isoforms of PLXNA-4 Myc with or without 3 nM SEMA3A stimulation for lhr. Cells are immunostained with anti-Myc (green) and AT8 (red). Scale bar represents 10 tm. *p<0.05, **p<0.01, and ***p<0.001, as determined by ANOVA and Tukey post hoc.

FIGS. 3A-3B show RNA expression of PLXNA4 isoforms. (3A) Normalized mRNA expression of full length isoform 1 (TS1) and shorter isoform 3 (TS3) in brain tissue obtained from five control subjects (mean age at death=80.4+6.3 years) without clinical or pathological evidence of AD and nine patients with autopsy-confirmed AD (mean age at death=91.0+2.2 years). Both isoforms are significantly higher in AD cases than controls (see Table 4). Among controls expression of TS3 is significantly greater (P=0.001). (3B) Normalized mRNA expression levels of TS1 and TS3 according to presence or absence of the AD-associated PLXNA4 SNP rs1593222 risk allele (I) in serum from 116 control subjects. Scale bar represents 10 tm. *p<0.05, **p<0.01, and ***p<0.001, as determined by ANOVA.

FIGS. 4A-4E show genetic findings in the PLXNA4 region. Regional association plots of genotyped and imputed SNPs from the FHS (4A) and NIA-LOAD (4B) datasets, and in meta-analysis (4C). Most significant SNPs in the FHS (rs277470) and NIA-LOAD (rs12539196) datasets are indicated by purple diamonds. P-values are expressed as −log 10(P) (y-axis) for every tested SNP ordered by chromosomal location (x-axis). Estimates of linkage disequilibrium (r²) of SNPs in this region with the top SNP computed using 1000 Genomes (hg19/November 2010EUR) are shown as orange circles for r²≧0.8, yellow circles for 0.5≦r²<0.8, light blue circles for 0.2≦r²<0.5, and blue circles for r²<0.2. Genomic structure of PLXNA4 was determined using the NCBI database (Build 37.1). (4D) Relative position of the most significantly associated SNPs in FHS and NIA-LOAD datasets in the three validated transcripts (TS1, TS2, and TS3). Exons are denoted with horizontal bars. (4E) Diagrams of functional domains encoded by amino acids from the full-length (TS1) and the shorter (TS2 and TS3) transcripts. SEMA: sema_plexinA1 interacting module (exon 1-4 in TS1 and exon 1-3 in TS2 and TS3); PSI: plexin repeat (exon 4-11 in TS1); IPT: three repeats of the binding domains of plexins and cell surface receptors (exon 11-17 n TS1); PCSR: binding domain of plexins and cell surface receptors (PCSR) and related proteins (exon 17-19 in TS1); TM: transmembrane region (exon 19 in TS1); CYTO: cytoplasmic domain (exon 20-31 in TS1).

FIG. 5 shows linkage disequilibrium (LD, D′) of top ranked SNPs. LD was calculated in 1000 genomes data from CEU for Caucasian, AFR for African American, and ASN for Japanese populations. Top-ranked SNPs from the PLXNA4 gene: rs277470, rs277472, and rs277484 in FHS; rs12539196 in NIA-LOAD; rs10273901 in ADGC-EA; rs75460865 in ADGC-AA; rs13232207 in ADGC-JPN. The top SNP from the ADGC-AA was monomorphic in both EUR and ASN populations. Five top-ranked SNPs (rs10273901, rs75460865, rs277470, rs277472, and rs277484) from the Caucasian and African American samples were monomorphic in the ASN sample. The top ranked SNPs are located in the SEMA domain, except rs10273901 and rs13232207 which are located in the cytoplasmic domain.

### DETAILED DESCRIPTION

Embodiments of the various aspects disclosed herein are generally related to assays, methods and systems for identifying a subject with an increased risk for late-onset AD. In one embodiment, the assays, methods and systems are directed to detection of single nucleotide polymorphisms (SNPs) associated with late-onset AD in a biological sample of a subject. In another embodiment, the assays, methods and systems are directed to determination of the expression level of the corresponding SNP gene product in a biological sample of a subject. Another aspect of the invention is directed to methods and pharmaceutical compositions for therapeutic treatment of AD, e.g., by administering aTS1 PLXNA4 inhibitory agent to a subject diagnosed with or at risk of AD.

In one aspect, the disclosure provides a method for inhibiting progression of AD. The method comprising administering having or at risk for Alzheimer's disease a therapeutically effective amount of a TS1 PLXNA4 inhibitory agent. In some embodiments, the method further comprises assaying a biological sample from the subject for the presence or absence of one or more AD risk associated SNPs before onset of said administering.

The disclosure also provides a method for inhibiting or reducing neurofibrillary tangles in the brain. The method comprising administering to a subject having or at risk for having neurofibrillary tangles in the brain a therapeutically effective amount of a TS1 PLXNA4 inhibitory agent. In some embodiments, the method further comprises assaying a biological sample from the subject for the presence or absence of one or more AD risk associated SNPs before onset of said administering.

In another aspect, the disclosure provides a method for inhibiting or reducing tau phosphorylation in the brain. The method comprising administering to a subject a therapeutically effective amount of a TS1 PLXNA4 inhibitory agent. In some embodiments, the method further comprises assaying a biological sample from the subject for the presence or absence of one or more AD risk associated SNPs before onset of said administering.

As used herein, a TS1 PLXNA4 inhibitory agent, refers to an agent that can inhibit expression and/or activity of the TS1 PLXNA4 isoform. In some embodiments, a TS1 PLXNA4 inhibitory agent can be specific for the TS1 isoform. In some embodiments, a TS1 PLXNA4 inhibitory agent also has some activity against other PLXNA4 isoforms, such as the TS2 and TS2 PLXNA4 isoforms, as described herein.

The term “agent” or “compound” as used herein, in regard to, for example, a TS1 PLXNA4 inhibitory agent, refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, minibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of, for example, a TS1 PLXNA4 transcript, within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

In some embodiments, the TS1 PLXNA4 inhibitory agent is an oligonucleotide. In some embodiments, the TS1 PLXNA4 inhibitory agent is an anti-miR, antagomir, antisense oligonucleotide, ribozyme, aptamer, siRNA, shRNA, or RNAi agent.

In some embodiments, the TS1 PLXNA4 inhibitory agent is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition to, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

In some embodiments, the TS1 PLXNA4 inhibitory agent is RNA-interference or RNA interference molecule, including, but not limited to double-stranded RNA, such as siRNA, double-stranded DNA or single-stranded DNA. In some embodiments, an anti-miR-130/301 agent is a single-stranded RNA (ssRNA), a form of RNA endogenously found in eukaryotic cells as the product of DNA transcription. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

Numerous specific siRNA molecules have been designed that have been shown to inhibit gene expression (Ratcliff et al. Science 276:1558-1560, 1997; Waterhouse et al. Nature 411:834-842, 2001). In addition, specific siRNA molecules have been shown to inhibit, for example, HIV-1 entry to a cell by targeting the host CD4 protein expression in target cells thereby reducing the entry sites for HIV-1 which targets cells expressing CD4 (Novina et al. Nature Medicine, 8:681-686, 2002). Short interfering RNA have further been designed and successfully used to silence expression of Fas to reduce Fas-mediated apoptosis in vivo (Song et al. Nature Medicine 9:347-351, 2003).

It has been shown in plants that longer, about 24-26 nt siRNA, correlates with systemic silencing and methylation of homologous DNA. Conversely, the about 21-22 nt short siRNA class correlates with mRNA degradation but not with systemic signaling or methylation (Hamilton et al. EMBO J. 2002 Sep. 2; 21(17):4671-9). These findings reveal an unexpected level of complexity in the RNA silencing pathway in plants that may also apply in animals. In higher order eukaryotes, DNA is methylated at cytosines located 5′ to guanosine in the CpG dinucleotide. This modification has important regulatory effects on gene expression, especially when involving CpG-rich areas known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosomes of females. Aberrant methylation of normally unmethylated CpG islands has been documented as a relatively frequent event in immortalized and transformed cells and has been associated with transcriptional inactivation of defined tumor suppressor genes in human cancers. In this last situation, promoter region hypermethylation stands as an alternative to coding region mutations in eliminating tumor suppression gene function (Herman, et al.). The use of siRNA molecules for directing methylation of a target gene is described in U.S. Provisional Application No. 60/447,013, filed Feb. 13, 2003, referred to in U.S. Patent Application Publication No. 20040091918.

It is also known that the RNA interference does not have to match perfectly to its target sequence. Preferably, however, the 5′ and middle part of the antisense (guide) strand of the siRNA is perfectly complementary to the target nucleic acid sequence.

The RNA interference-inducing molecule functioning as TS1 PLXNA4 inhibitory agent includes RNA molecules that have natural or modified nucleotides, natural ribose sugars or modified sugars and natural or modified phosphate backbone. Accordingly, the RNA interference-inducing molecules functioning as anti-miR-130/301 agen includes, but are not limited to, unmodified and modified double stranded (ds) RNA molecules including short-temporal RNA (stRNA), small interfering RNA (siRNA), short-hairpin RNA (shRNA), microRNA (miRNA), and double-stranded RNA (dsRNA), (see, e.g. Baulcombe, Science 297:2002-2003, 2002). The dsRNA molecules, e.g. siRNA, also may contain 3′ overhangs, preferably 3′UU or 3′TT overhangs. In one embodiment, the siRNA molecules do not include RNA molecules that comprise ssRNA greater than about 30-40 bases, about 40-50 bases, about 50 bases or more. In one embodiment, the siRNA molecules have a double stranded structure. In one embodiment, the siRNA molecules are double stranded for more than about 25%, more than about 50%, more than about 60%, more than about 70%, more than about 80% or more than about 90% of their length.

Anti-miRs, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the compositions and methods disclosed herein. Anti-miRs can be used to efficiently silence endogenous miRNAs by forming duplexes comprising the anti-miR and endogenous miRNA, thereby preventing miRNA-induced gene silencing.

In some embodiments, the TS1 PLXNA4 inhibitory agent is an antagomir. Antagomirs are oligonucleotide anti-miRs that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In some embodiments, antagomir comprises a 2′-O-methylmodification at all nucleotides, a cholesterol moiety at 3′-end, two phsophorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

In some embodiments, the TS1 PLXNA4 inhibitory agent is ribozyme. In some embodiments, the anti-miR-130/301 agent is ribozyme that cleaves the target microRNA. Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Methods of producing a ribozyme targeted to any target sequence are known in the art. Ribozymes can be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to up-regulate genes that are suppressed by the binding of a transcription factor. Examples of the utilization of decoy oligonucleotides can be found in Mann et al., J. Clin. Invest., 2000, 106: 1071-1075, which is expressly incorporated by reference herein, in its entirety. Thus, in some embodiments, the anti-miR-130/301 agent is a decoy oligonucleotide.

In some embodiments, the TS1 PLXNA4 inhibitory agent comprises a sequence substantially complimentary to at least 15 (e.g., 15, 16, 17, 18, 19, 20, 21, 22, 23, 24, 25, 26, 27, 28, 29, or 30) contiguous nucleotides of SEQ ID NO: 1. In some embodiments, the TS1 PLXNA4 inhibitory agent comprises a sequence substantially identical to at least 15 (e.g., 15, 16, 17, 18, 19, 20, 21, 22, 23, 24, 25, 26, 27, 28, 29, or 30) contiguous nucleotides of SEQ ID NO: 1

In some embodiments, the TS1 PLXNA4 inhibitory agent comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the TS1 PLXNA4 inhibitory agent comprises the nucleotide sequence selected from: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the TS1 PLXNA4 inhibitory agent is an antibody or fragment thereof. The terms “antibody” and “antibodies” include polyclonal antibodies, monoclonal antibodies, humanized or chimeric antibodies, single chain Fv antibody fragments, Fab fragments, and F(ab)₂ fragments. Without limitations, the antibody can be a recombinant antibody, humanized antibody, chimeric antibody, modified antibody, monoclonal antibody, polyclonal antibody, miniantibody, dimeric miniantibody, minibody, diabody or tribody or antigen-binding variants, analogues or modified versions thereof. Antibodies having specific binding affinity for PLXNA4 can be produced through standard methods. Alternatively, antibodies may be commercially available, for example, from R&D Systems, Inc., Minneapolis, Minn.

As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies. Unless it is specifically noted, as used herein a “fragment thereof” in reference to an antibody refers to an immunospecific fragment, i.e., an antigen-specific or binding fragment.

Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., Monoclonal Antibodies and Cancer Therapy, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

Polyclonal antibodies are heterogeneous populations of antibody molecules that are specific for a particular antigen, which are contained in the sera of the immunized animals. Polyclonal antibodies are produced using well-known methods. A chimeric antibody is a molecule in which different portions are derived from different animal species, such as those having a variable region derived from a murine monoclonal antibody and a human immunoglobulin constant region. Chimeric antibodies can be produced through standard techniques. Antibody fragments that have specific binding affinity for a component of the complex can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246: 1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

In some embodiments, the antibody or antigen-binding fragment thereof is murine. In some embodiments, the antibody or antigen-binding fragment thereof is from mice. In some embodiments, the antibody or antigen-binding fragment thereof is from rat. In other embodiments, the antibody or antigen binding fragment thereof is human. In some embodiments the antibody or antigen-binding fragment thereof is recombinant, engineered, humanized and/or chimeric.

As used herein, the terms “treatment” and “treating,” with respect to treatment of AD, means preventing the progression for the disease, or altering the course of the disorder (for example, but not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of AD treatment, therapeutic treatment refers to reducing the cognitive deterioration in a subject and/or inhibiting or reducing the level of Aβ in the brain of a subject that is already inflicted with AD. Measurable lessening includes any statistically significant decline in a measurable marker or symptom, such as measuring Aβ in the brain by PET scan, or assessing the cognitive improvement with neuropsychological tests such as verbal and perception after treatment.

The term “therapeutically effective amount” refers to an amount effective, at dosages and for periods of time necessary, to achieve the desired therapeutic result, e.g., a diminishment or prevention of effects associated with various disease states or conditions, such as reduce a symptom of an Alzheimer's disease in the subject. The term “therapeutically effective amount” refers to an amount of, for example, a TS1 PLXNA4 inhibitory agent, as disclosed herein, effective to treat or prevent a disease or disorder in a mammal, preferably a human. In the case of treatment of Alzheimer's, a therapeutically effective amount may alleviate one or more symptoms associated with the disease including increasing long-term memory, for example.

As used herein, the terms “administering,” and “introducing” are used interchangeably herein and refer to the placement of the agents, such as a TS1 PLXNA4 inhibitory agent, into a subject by a method or route which results in at least partial localization of a TS1 PLXNA4 inhibitory agent at a desired site such that desired effect is produced, such as intracranially to brain or specific areas of brain. Stereotactic means can be used to guide intracranial administration if desired. Routes of administration suitable for the methods of the invention include both local and systemic administration. Generally, local administration results in more of the composition being delivered to a specific location as compared to the entire body of the subject, whereas, systemic administration can result in delivery to essentially the entire body of the subject. However, it is envisioned that chemotropic property of NSCs can guide the cells to a specific location with a tissue injury, e.g., brain, even with systemic administration. The agent can be administered by any appropriate route which results in an effective treatment in the subject, including, but not limited to, oral or parenteral routes, including intravenous, intramuscular, subcutaneous, transdermal, and nasal administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub-capsular, subarachnoid, intraspinal, intracerebro spinal, and intrasternal injection and infusion.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of a TS1 PLXNA4 inhibitory agent such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation. Pharmaceutically acceptable carriers are well known to those of skill in the art.

In some embodiments, the disclosure also provides assays to identify a subject with an increased risk for developing late onset AD. In one embodiment, the assay comprises or consists essentially of a system for transforming and identifying at least one nucleic acid polymorphism in a SNP locus described herein in a biological sample of a subject, and a system for computing the likelihood of the subject getting late onset AD on the basis of comparison of the identified nuclei acid at the SNP locus against the AD risk associated alleles described herein. If the computing or comparison system, which can be a computer implemented system, indicates that at least one of the allele at the SNP locus is identical to the corresponding AD risk associated allele, the subject from which the sample is collected can be diagnosed with increased susceptibility for late onset AD.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

In one aspect, provided herein is an assay. The assay comprising subjecting a test sample from a subject, e.g., a human subject, to at least one genotyping assay that determines the genotypes of at least one (e.g., one, tow, three, four, five, six, seven, eight, nine, ten or more) loci selected from SNP rs277470, rs277472, rs277473, rs277474, rs277476, rs277477, rs277478, rs277479, rs277480, rs277481, rs277483, rs277484, rs10234979, rs9641933, rs10225863, rs7799929, rs9656410, rs11764790, rs4731860, rs11763817, rs13231950, rs11773243, rs17166338, rs11773724, rs17818325, rs10236235, rs965800, rs10244929, rs4481530, rs1593222, rs1424590, rs1364505, rs6959579, rs11761937, and any combinations thereof; and determining the genotype of said at least one at least one (e.g., one, tow, three, four, five, six, seven, eight, nine, ten or more) loci.

In some embodiments, the loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; and (iii) SNP3, wherein SNP3 identified by rs11761937 on SEQ ID NO: 1, wherein the SEQ ID NO. 1 is a portion of genomic nucleic acid sequence of PLXNA4.

In some embodiments, the loci are further selected from: (i) SNP4, wherein SNP4 is identified by rs1593222 on SEQ ID NO: 1, wherein the SEQ ID NO. 1 is a portion of genomic nucleic acid sequence of PLXNA4; (ii) SNP5, wherein SNP4 is identified by rs6959579 on SEQ ID NO: 1, wherein the SEQ ID NO. 1 is a portion of genomic nucleic acid sequence of PLXNA4; and (iii) SNP6, wherein SNP4 is identified by rs17166339 on SEQ ID NO: 1, wherein the SEQ ID NO. 1 is a portion of genomic nucleic acid sequence of PLXNA4.

In some embodiments, the assay further comprises selecting a treatment regimen that comprises a therapeutically effective amount of a TS1 PLXNA4 inhibitory agent when at least one (e.g., one, two, three or more) of the following combinations of SNPs is determined to be present: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4.

In another aspect, the disclosure provides a method for treating a subject, e.g. a human subject having or at risk for Alzheimer's disease. The method comprising administering a therapeutically effective amount of a TS1 PLXNA4 inhibitory agent to the subject which is determined to carry at least one (e.g., one, two, three or more) SNPs selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4.

In yet another aspect, the disclosure provides a method for selecting a subject having or at risk for AD, wherein the subject is susceptible to treatment with a TS1 PLXNA4 inhibitory agent. The method comprising: (a) contacting a biological sample with at least one (e.g., one, two, three, four or more) oligonucleotide capable of interrogating whether or not the biological sample comprises one or more of the single nucleotide polymorphisms (SNPs) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (b) identifying Alzheimer's disease in the subject as susceptible for treatment with a TS1 PLXNA4 inhibitory agent when at least one (e.g., one, two, or three) of the SNPs of (i)-(iii) is detected in the biological sample, and identifying Alzheimer's disease in the subject as poorly or non-responsive to treatment with the TS1 PLXNA4 inhibitory agent when none of the SNPs of (i)-(iii) is detected in the biological sample.

In one aspect, the disclosure provide an assay comprising: (a) contacting a biological sample obtained from a subject with a detectable antibody specific for PLXNA4 or detectable nucleic acid complementary to at least part of PLXNA4, e.g., SNP rs277472, SNP rs10236235, and/or SNP rs11761937locus; (b) washing the sample to remove unbound antibody or unbound nucleic acid; (c) measuring the intensity of the signal from the bound, detectable antibody or bound detectable nucleic acid; (d) comparing the measured intensity of the signal with a reference value and if the measured intensity is normal and/or increased relative to the reference value; the subject is identified as having or at risk for AD.

Another aspect of the present invention relates to a system for obtaining data from at least one test sample obtained from at least one subject, the system comprising: (a) a determination module configured to receive said at least one test sample and perform at least one analysis on said at least one test sample to determine the presence or absence of at least one of the following conditions: (i) the level of expression or amount of PLXNA4 (e.g., PLXNA4 isoform TS1 or TS3) is higher than a pre-determined level; (ii) at least one copy of a single nucleotide polymorphism (SNP) selected from: (1) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (2) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (3) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (4) any combinations thereof; (b) a storage device configured to store data output from said determination module; and (c) a display module for displaying a content based in part on the data output from said determination module, wherein the content comprises a signal indicative of the presence of at least one of these conditions determined by the determination module, or a signal indicative of the absence of at least one of these conditions determined by the determination module.

In some embodiments, the content displayed from the display module of the system as disclosed herein can further comprise a signal indicative of the subject being recommended to receive a particular treatment regimen, for example, if the subject has one or more of the above conditions, a signal is produced to recommend the subject be administered an AD therapy, for example, but not limited to, a TS1 PLXNA4 inhibitory agent.

In some embodiments, the subject is recommended for AD therapy, e.g., a treatment with a composition comprising a TS1 PLXNA4 inhibitory agent, where the content from the display module produces a signal indicative of at least one of: (a) increased expression level or amount of TS1 or TS3 PLXNA4 isoform; and (b) presence of at least one AD risk associated SNP as disclosed herein.

In some embodiments, a subject is not recommended for AD therapy, e.g., a treatment with a composition comprising a TS1 PLXNA4 inhibitory agent, where the content from the display module produces a signal indicative of at least one of: (a) lower or reduced expression level or amount of TS1 or TS3 PLXNA4 isoform; and (b) absence of at least one AD risk associated SNP as disclosed herein.

In another aspect, the disclosure provides a method of determining if a subject is responsive to a TS1 PLXNA4 inhibitory agent. The method comprising assaying a blood sample for the presence of at least one (e.g., one, two, or three) of: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4.

### SNPs, Polymorphisms and Alleles

The genomes of all organisms undergo spontaneous mutation in the course of their continuing evolution, generating variant forms of progenitor genetic sequences (Gusella, Ann. Rev. Biochem. 55, 831-854 (1986)). The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. The SNP position (interchangeably referred to herein as SNP, SNP site, SNP allele or SNP locus) is usually preceded by and followed by highly conserved sequences of the allele (e.g., sequences that vary in less than 1/100 or 1/1000 members of the populations). An individual can be homozygous or heterozygous for an allele at each SNP position. A SNP can, in some instances, be referred to as a “cSNP” to denote that the nucleotide sequence containing the SNP is an amino acid coding sequence.

A SNP can arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP can also be a single base insertion or deletion variant referred to as an “in/del” (Weber et al., “Human diallelic insertion/deletion polymorphisms”, Am J Hum Genet October 2002; 71(4):854-62).

A synonymous codon change, or silent mutation/SNP (the terms “SNP” and “mutation” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

A major database of human SNPs is maintained at NCBI as dbSNP, and it contains data for unique human SNPs consisting of 1.1×10⁸ submitted SNP (identified by an “ss” number) and 2.4×10⁷ reference SNP (identified by an “rs” number), as of Build History 131: human_9606 based on GRCh37 available from the NCBI website. The rs numbers are unique, do not change and allow analysis of the particularly identified SNP in any genetic sample. Throughout the specification, the SNPs described herein are identified by an “rs” number. One of skill in the art will be able to determine the position of a specific SNP within a respective chromosome.

The most common type of SNP in humans has alleles A and G. Since DNA is a double helix, the opposite strand has alleles T and C. So an A/G SNP can also be described as a T/C SNP, depending upon orientation. The distribution of the types of SNPs in humans was estimated as follows: 63% A/G (and T/C), 17% A/C (and T/G), 8% CG, 4% AT, and 8% insertion/deletions (Miller, R. D., P. Taillon-Miller, and P. Y. Kwok. 2001. Regions of Low Single-Nucleotide Polymorphism Incidence in Human and Orangutan Xq: Deserts and Recent Coalescences. Genomics 71: 78-88).

While a SNP could conceivably have three or four alleles, nearly all SNPs have only two alleles. Analysis of the SNPs identified in this study all rely on the two alleles that are listed in connection with each SNP. For example, one of the AD risk associated SNP described herein, rs277472 is indicated to have two alleles, A or C. The presence of an allele A at the rs277472 locus indicates an increased risk for AD.

An association study of a SNP and a specific disorder involves determining the presence or frequency of the SNP allele in biological samples from subjects with the disorder of interest, such as Alzheimer's disease, and comparing the information to that of controls (i.e., individuals who do not have the disorder; controls can be also referred to as “healthy” or “normal” individuals) who are preferably of similar age and race. The appropriate selection of patients and controls is important to the success of SNP association studies. Therefore, a pool of individuals with well-characterized phenotypes is desirable. Association studies can be conducted within the general population and are not limited to studies performed on related individuals in affected families (linkage studies).

A SNP can be screened in any biological sample obtained from an individual or a subject diagnosed with or at risk of a disease or disorder, e.g., Alzheimer's disease. If an allele herein discovered as an AD risk allele is identified, the subject can be identified as at greater risk of developing AD than a subject who is not carrying that alleles.

Particular SNP alleles, sometimes referred to as polymorphisms or polymorphic alleles, of the present invention can be associated with an increased risk of developing AD. In some embodiments the AD is late-onset form. Mutations or alleles identifying a subject with an increased risk of developing a disorder, for example, late onset AD, are also referred to as “susceptibility” alleles, or mutations.

Those skilled in the art will readily recognize that nucleic acid molecules can be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular SNP position, SNP allele, or nucleotide sequence. Probes and primers can be designed to hybridize to either strand and SNP genotyping methods disclosed herein can generally target either strand. Accordingly, the claims are intended to cover analysis of the opposite strand as well. For the opposite-strand analysis.

Identification method of SNPs can be of either a positive-type (inclusion of an allele) or a negative-type (exclusion of an allele). Positive-type methods determine the identity of a nucleotide contained in a polymorphic site, whereas negative-type methods determine the identity of a nucleotide not present in a polymorphic site. Thus, a wild-type site can be identified either as wild-type or not mutant. For example, at a biallelic polymorphic site where the wild-type allele contains a cytosine and the mutant allele contains adenine, a site can be positively determined to be either adenine or cytosine or negatively determined to be not adenine (and thus cytosine) or not cytosine (and thus adenine).

In one aspect, the nucleic acid sequences of the gene's allelic variants, or portions thereof, can be the basis for probes or primers, e.g., in methods for determining the identity of the allelic variant of the polymorphic region. Thus, in one embodiment, nucleic acid probes or primers can be used in the methods of the present invention to determine whether a subject is at risk of developing disease such as Alzheimer's disease. One of skill in the art can readily access the nucleic acid sequences spanning the SNPs described herein through the NCBI dbSNP database with the “rs” number uniquely assigned to each SNPs described herein. Thus, a skilled artisan can readily design and optimize primers or probes based on the flanking sequences of the SNP loci described herein.

The polymorphisms of PLXNA4 disclosed herein can be detected directly or indirectly using any of a variety of suitable methods including fluorescent polarization, mass spectroscopy, and the like. Suitable methods comprise direct or indirect sequencing methods, restriction site analysis, hybridization methods, nucleic acid amplification methods, gel migration methods, the use of antibodies that are specific for the proteins encoded by the different alleles of the polymorphism, or by other suitable means. Alternatively, many such methods are well known in the art and are described, for example in T. Maniatis et al., Molecular Cloning, a Laboratory Manual, 2nd Edition, Cold Spring Harbor Press, Cold Spring Harbor, N.Y. (1989), J. W. Zyskind et al., Recombinant DNA Laboratory Manual, Academic Press, Inc., New York (1988), and in R. Elles, Molecular Diagnosis of Genetic Diseases, Humana Press, Totowa, N.J. (1996), and Mamotte et al, 2006, Clin Biochem Rev, 27; 63-75) each herein incorporated by reference.

Methods to measure gene expression products associated with AD risk associated SNPs described herein are well known to a skilled artisan. Such methods to measure gene expression products, e.g., protein level, include ELISA (enzyme linked immunosorbant assay), western blot, immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques, particularly useful are methods that detect the allelic variant of a peptide expressed in a subject and methods which detect fragments of a peptide in a sample.

Without limitations, any approach that detects mutations or polymorphisms in a gene can be used, including but not limited to single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes of the present invention (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

In some embodiments, the gene expression products as described herein can be determined by determining the level of messenger RNA (mRNA) expression of genes associated with SNPs described herein (e.g., PLXNA 4). Such molecules can be isolated, derived, or amplified from a biological sample, such as body fluids. Detection of mRNA expression is known by persons skilled in the art, and comprise, for example but not limited to, PCR procedures, RT-PCR, Northern blot analysis, differential gene expression, RNA protection assay, microarray analysis, hybridization methods etc.

Nucleic acid or ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, an allelic discrimination method can be used for identifying the genotype of SNPs described herein. In some embodiments, the allelic discrimination method involves use of a first oligonucleotide probe which anneals with a target portion of the individual's genome. Because the nucleotide residue at this position differs, the first probe is completely complementary to only one of the two alleles. Alternatively, a second oligonucleotide probe can also be used which is completely complementary to the target portion of the other of the two alleles. The allelic discrimination method also involves use of at least one, and preferably a pair of amplification primers for amplifying a reference region, for example, at least a portion of the flanking region including the SNP locus of interest.

The probe in some embodiments is a DNA oligonucleotide having a length in the range from about 20 to about 40 nucleotide residues, preferably from about 20 to about 30 nucleotide residues, and more preferably having a length of about 25 nucleotide residues. In one embodiment, the probe is rendered incapable of extension by a PCR-catalyzing enzyme such as Taq polymerase, for example by having a fluorescent probe attached at one or both ends thereof. Although non-labeled oligonucleotide probes can be used in the kits and methods of the invention, the probes are preferably detectably labeled. Exemplary labels include radionuclides, light-absorbing chemical moieties (e.g. dyes), fluorescent moieties, and the like. Preferably, the label is a fluorescent moiety, such as 6-carboxyfluorescein (FAM), 6-carboxy-4,7,2′,7′-tetrachlorofluoroscein (TET), rhodamine, JOE (2,7-dimethoxy-4,5-dichloro-6-carboxyfluorescein), HEX (hexachloro-6-carboxyfluorescein), or VIC.

In some embodiments, the probe can comprise both a fluorescent label and a fluorescence-quenching moiety such as 6-carboxy-N,N,N′,N′-tetramethylrhodamine (TAMRA), or 4-(4′-dimethlyaminophenylazo)benzoic acid (DABCYL). When the fluorescent label and the fluorescence-quenching moiety are attached to the same oligonucleotide and separated by no more than about 40 nucleotide residues, and preferably by no more than about 30 nucleotide residues, the fluorescent intensity of the fluorescent label is diminished. When one or both of the fluorescent label and the fluorescence-quenching moiety are separated from the oligonucleotide, the intensity of the fluorescent label is no longer diminished. In some embodiments, the probe of the present invention has a fluorescent label attached at or near (i.e. within about 10 nucleotide residues of) one end of the probe and a fluorescence-quenching moiety attached at or near the other end. Degradation of the probe by a PCR-catalyzing enzyme releases at least one of the fluorescent label and the fluorescence-quenching moiety from the probe, thereby discontinuing fluorescence quenching and increasing the detectable intensity of the fluorescent labels. Thus, cleavage of the probe (which, as discussed above, is correlated with complete complementarity of the probe with the target portion) can be detected as an increase in fluorescence of the assay mixture.

If different detectable labels are used, more than one labeled probe can be used, and therefore polymorphisms can be performed in multiplex. For example, the assay mixture can contain a first probe which is completely complementary to the target portion of a first AD associated SNP and to which a first label is attached, and a second probe which is completely complementary to the target portion of a second AD risk associated SNP. When two probes are used, the probes are detectably different from each other, having, for example, detectably different size, absorbance, excitation, or emission spectra, radiative emission properties, or the like. For example, a first probe can be completely complementary to the target portion of the polymorphism and have FAM and TAMRA attached at or near opposite ends thereof. The first probe can be used in the method of the present invention together with a second probe which is completely complementary to the target portion of another AD risk associated and has TET and TAMRA attached at or near opposite ends thereof. Fluorescent enhancement of FAM (i.e. effected by cessation of fluorescence quenching upon degradation of the first probe by Taq polymerase) can be detected at one wavelength (e.g. 518 nanometers), and fluorescent enhancement of TET (i.e. effected by cessation of fluorescence quenching upon degradation of the second probe by Taq polymerase) can be detected at a different wavelength (e.g. 582 nanometers). Using multiplexing methods, more than one SNP described herein can be detected, providing a better diagnosis and more reliable prediction of AD susceptibility in a subject.

In some embodiments, the probe comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the probe comprises the nucleotide sequence selected from: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

Another allelic discrimination method suitable for use in detection of SNPs employs “molecular beacons”. Detailed description of this methodology can be found in Kostrikis et al., Science 1998, 279:1228-1229, content of which is incorporated herein by reference in its entirety.

In some embodiments, the molecular beacon probe comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the molecular beacon probe comprises the nucleotide sequence selected from: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

The use of microarrays comprising a multiplicity of sequences is becoming increasingly common in the art. Accordingly, a microarray having at least one oligonucleotide probe, as described above, appended thereon, can be used for SNP genotyping.

In some embodiments, restriction enzymes can be utilized to identify variances or a polymorphic site using “restriction fragment length polymorphism” (RFLP) analysis (Lentes et al., Nucleic Acids Res. 16:2359 (1988); and C. K. McQuitty et al., Hum. Genet. 93:225 (1994)). In RFLP, at least one target polynucleotide is digested with at least one restriction enzyme and the resulting restriction fragments are separated based on mobility in a gel. Typically, smaller fragments migrate faster than larger fragments. Consequently, a target polynucleotide that contains a particular restriction enzyme recognition site will be digested into two or more smaller fragments, which will migrate faster than a larger fragment lacking the restriction enzyme site. Knowledge of the nucleotide sequence of the target polynucleotide, the nature of the polymorphic site, and knowledge of restriction enzyme recognition sequences guide the design of such assays. In another embodiment of the present invention, restriction site analysis of particular nucleotide sequence to identify a nucleotide at a polymorphic site is determined by the presence or absence of a restriction enzyme site. A large number of restriction enzymes are known in the art and, taken together, they are capable of recognizing at least one allele of many polymorphisms. However, such single nucleotide polymorphisms (SNPs) rarely result in changes in a restriction endonuclease site. Thus, SNPs are rarely detectable by restriction fragment length analysis.

A number of approaches use DNA ligase, an enzyme that can join two adjacent oligonucleotides hybridized to a DNA template. In Oligonucleotide ligaton assay (OLA) the sequence surrounding the mutation site is first amplified and one strand serves as a template for three ligation probes, two of these are ASO (allele-specific oligonucleotides) and a third common probe. Numerous approaches cane be used for the detection of the ligated products, for example the ASOs with differentially labeled with fluorescent of hapten labels and ligated products detected by fluorogenic of colorimetric enzyme-linked immunosorbant assays (Tobe et al, Nucleic Acid Res, 1996; 24; 3728-32). For electrophoresis-based systems, use of a morbidity modifier taqgs or variation in probe length coupled with fluorescence detection enables the multiplex genotyping of several single nucleotide substitutions in a single tube (Baron et al, 1997; Clinical Chem., 43, 1984-6). When used on arrays, ASOs can be spotted at specific locations or addresses on a chip, PCR amplified DNA can then be added and ligation to labeled oligonucleotides at specific addresses on the array measured (Zhong et al, Proc Natl Acad Sci 2003; 100, 11559-64).

Single base-extension or minisequencing involves annealing an oligonucleotide primer to the single strand of a PCR product and the addition of a single dideoxynucleotide by thermal DNA polymerase. The oligonucleotide is designed to be one base short of the mutation site. The dideoxynucleotide incorporated is complementary to the base at the mutation site. Approaches cans uses different fluorescent tags or haptens for each of the four different dideoxynucleotides (Pastinen et al, Clin Chem 1996, 42, 1391-7). The dideoxynucleotide differ in molecular weight and this is the basis for single-base extension methods utilizing mass-spectrometry, and genotyping based on the mass of the extended oligonucleotide primer, can be used, for example matrix-assisted laser adsorption/ionization time-of flight mass spectrometry or MALDI-TOF (Li et al, Electrophoresis, 1999,20; 1258-65), which is quantitative and can be used to calculate the relative allele abundance making the approach suitable for other applications such as gene dosage studies (for example for estimation of allele frequencies on pooled DNA samples).

Minisequencing or Microsequencing by MALDI-TOF can be performed by means known by persons skilled in the art. In a variation of the MALDI-TOF technique, some embodiments can use the Sequenom's Mass Array Technology (www.sequenom.com) (Sauser et al, Nucleic Acid Res, 2000, 28; E13 and Sauser et al, Nucleic Acid Res 2000, 28: E100). and also the GOOD Assay (Sauer S et al, Nucleic Acid Res, 2000; 28, E13 and Sauer et al, Nucleic Acid Res, 2000; 28:E100).

In some embodiments, variations of MALDI-TOF can be performed for analysis of variances in the genes associated with SNPs described herein. For example, MALDI and electrospray ionization (ESI) (Sauer S. Clin Chem Acta, 2006; 363; 93-105) is also useful with the methods of the present invention.

Allele-specific Amplification is also known as amplification refectory mutation system (ARMS) uses allele specific oligonucleotides (ASO) PCR primers and is an well established and known PCR based method for genotyping (Newton et al, J Med Genet, 1991; 28; 248-51). Typically, one of the two oligonucleotide primers used for the PCR binds to the mutation site, and amplification only takes place if the nucleotide of the mutation is present, with a mismatch being refractory to amplification. The resulting PCR Products can be analyzed by any means known to persons skilled in the art. In a variation of the approach, termed mutagenically separated PCR (MS-PCR) the two ARMS primer of different lengths, one specific for the normal gene and one for the mutation are used, to yield PCR procures of different lengths for the normal and mutant alleles (Rust et al, Nucl Acids Res, 1993; 21; 3623-9). Subsequent gel electrophoresis, for example will show at least one of the two allelic products, with normal, mutant or both (heterozygote) genes. A further variation of this forms the basis of the Masscode System™ (www.bioserve.com) which uses small molecular weight tags covalently attached through a photo-cleavable linker to the ARMS primers, with each ARMS primers labeled with a tag of differing weight (Kokoris et al, 2000, 5; 329-40). A catalogue of numerous tags allows simultaneous amplification/genotyping (multiplexing) of 24 different targets in a single PCR reaction. For any one mutation, genotyping is based on comparison of the relative abundance of the two relevant mass tags by mass spectrometry.

Normal or mutant alleles can be genotyped by measuring the binding of allele-specific oligonucleotides (ASO) hybridization probes. In such embodiments, two ASO probes, one complementary to the normal allele and the other to the mutant allele are hybridized to PCR-amplified DNA spanning the mutation site. In some embodiments, the amplified products can be immobilized on a solid surface and hybridization to radiolabelled oligonucleotides such as known as a ‘dot-blot’ assay. In alternative embodiments, the binding of the PCR products containing a quantifiable label (eg biotin or fluorescent labels) to a solid phase allele-specific oligonucleotide can be measured. Alternatively, for a reverse hybridixation assay, or “reverse dot-blot” the binding of PCR products containing a quantifiable label (for example but not limited to biotin or fluorescent labels) to a solid phase allele-specific oligonucleotide can be measured. In some embodiments, the use of microarrays comprising hundreds of ASO immobilized onto a solid support surfaces to form an array of ASO can also be used for large scale genotyping of multiple single polymorphisms simultaneously, for example Affymetrix GENECHIP® Mapping 10K Array, which can easily be performed by persons skilled in the art.

In some embodiments, the ASO comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the ASO comprises a nucleotide sequence selected from (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

Homogenous assays, also called “closed tube” arrays, genomic DNA and all the reagents required for the amplification and genotyping are added simultaneously. Genotyping can be achieved without any post-amplification processing. In some embodiments, one such homogenous assay is the 5′fluorogenic nuclease assay, also known as the TAQMAN® Assay (Livak et al, Genet Anal, 1999; 14:143-9) and in alternative embodiments Melting curve analyses of FRET probes are used. Such methods are carried out using “real-time” theromcyclers, and utilize two dual-labeled ASO hybridization probes complementary to normal and mutant alleles, where the two probes have different reported labels but a common quencher dye. In such embodiments, the changes in fluorescence characteristics of the probes upon binding to PCR products of target genes during amplification enables “real-time” monitoring of PCR amplification and differences in affinity of the fluorogenic probes for the PCR products of normal and mutant genes enables differentiation of genotypes. The approach uses two dual-labeled ASO hybridization probes complementary to the mutant and normal alleles. The two probes have different fluorescent reported dyes but a common quencher dye. When intact, the probes do not fluoresces due to the proximity of the reporter and quencher dyes. During annealing phase of PCR, two probes compete for hybridization to their target sequences, downstream of the primer sites and are subsequently cleaved by 5′ nuclease activity of Thermophilis aquaticus (Taq) polymerase as the primer is extended, resulting in the separation of the reporter dyes from the quencher. Genotyping is determined by measurement of the fluorescent intensity of the two reporter dyes after PCR amplification. Thus, when intact the probes do not fluoresce due to the proximity of the quencher dyes, whereas during the annealing phase of the PCR the probes compete for hybridization of the target sequences and the separation of one of the probes from the quencher which can be detected.

Melting-curve analysis of FRET hybridization is another approach useful in the method of the invention. Briefly, the reaction includes two oligonucleotide probes which when in close proximity forms a fluorescent complex, where one probe often termed the “mutant sensor” probe is designed to specifically hybridize across the mutation site and the other probe (often referred to as the “anchor probe”) hybridizes to an adjacent site. Fluorescent light is emitted by the “donor” excites the “acceptor” fluorophore creasing a unique fluorogenic complex, which only forms when the probes bind to adjacent sites on the amplified DNA. The “sensor” probe is complementary to either the normal or the mutant allele. Once PCR is complete, heating of the sample through the melting temperatures of the probe yields a fluorescent temperature curve which differs for the mutant and normal allele.

A variation of the FRET hybridization method is the LCGREEN™ method, which obviates the requirement for fluorescent labeled probes altogether. LCGREEN™ is a sensitive highly fluorogenic double-stranded DNA (dsDNA) binding dye that is used to detect the dissociation of unlabelled probes (Liew et al, Clin Chem, 2004; 50; 1156-64 and Zhou et al, Clin Chem, 2005; 51; 1761-2). The method uses unlabeled allele-specific oligonucleotides probes that are perfectly complementary either to the mutant or normal allele, and the mismatch of the ASO/template double strand DNA complex results in a lower melting temperature and an earlier reduction in fluorescent signal form the dsDNA binding dye with increasing temperature.

The OLA can also be used for FRET Probes (Chen et al, 1998; 8:549-56), for example, the PCR/ligation mixture can contain PCR primers, DNA polymerase without 5′ nuclease activity, thermal stable DNA ligase and oligonucleotides for the ligation reaction. The ligation of the allele-specific oligonucleotides have a different acceptor fluorophore and the third ligation oligonucleotide, which binds adjacently to the ASO has a donor fluorophore, and the three ligation oligonucleotides are designed to have a lower melting temperature for the PCR primers to prevent their interference in the PCR amplification. Following PCR, the temperature is lowered to allow ligation to proceed, which results in FRET between the donor and acceptor dyes, and alleles can be disconcerted by comparing the fluorescence emission of the two dyes.

Alternatives to homogenous PCR- and hybridization-based techniques are also encompassed. For example, molecular beacons (Tyagi et al, Nat Biotech, 1998, 16: 49-53) and SCORPION® probes (Thelwell et al, Nucleic Acid Res, 2000; 28; 3752-610).

The OLA can also be performed by the use of FRET probes (Chen et al, Genome Res, 1998; 8: 549-56). In such an embodiment, the PCR/ligation mix contains PCR primers, a thermostable DNA polymerase without 5′ exonuclease activity (to prevent the cleavage of ligation probes during the ligation phase), a thermostable DNA ligase as well as the oligonucleotides for the ligation reaction. The ligation of the ASO each have a different acceptor flurophore and the third ligation oligonucleotide which binds adjacently to the ASO has a donor flurophore. The three ligation oligonucleotides are designed to have a lower melting temperature than the annealing temperature for the PCR primers to prevent their interference in PCR amplification. Following PCR, the temperature is lowered to allow ligation to proceed. Ligation results in FRET between donor and acceptor dyes, and alleles can be discerned by comparing the fluorescence emission of the two dyes.

Further, variations of the homogenous PCR- and hybridization based techniques to detect polymorphisms are also encompassed in the present invention. For example, the use of Molecular Beacons (Tyagi et al, Nat Biotech 1998; 16; 49-53) and SCORPION® Probes (Thelwell et al, Nucleic Acid Res 2000; 28; 3752-61). Molecular Beacons are comprised of oligonucleotides that have fluorescent reporter and dyes at their 5′ and 3′ ends, with the central portion of the oligonucleotide hybridizing across the target sequence, but the 5′ and 3′ flanking regions are complementary to each other. When not hybridized to their target sequence, the 5′ and 3′ flanking regions hybridize to form a stem-loop structure, and there is little fluorescence because of the proximity of the reported and the quencher dyes. However, upon hybridization to their target sequence, the dyes are separated and there is a large increase in the fluorescence. Mismatched probe-target hybrids dissociate at substantially lower temperatures than exactly matched complementary hybrids. There are a number of variations of the “molecular Beacon” approach. In some embodiments, such a variation includes use of SCORPION® Probes which are similar but incorporate a PCR primer sequence as part of the probe (Thelwell et al, Nucleic Acid Res 2000; 28; 3752-61). In another variation, ‘duplex’ format gives a better fluorescent signal (Solinas et al, Nucleic Acid Res, 2001, 29; E96).

In another embodiment, polymorphisms can be detected by genotyping using a homogenous or real-time analysis on whole blood samples, without the need for DNA extraction or real-time PCR. Such a method is compatible with FRET and TAQMAN® (Castley et al, Clin Chem, 2005; 51; 2025-30) enabling extremely rapid screening for the particular polymorphism of interest.

In Fluorescent Polarization (FP), the degree to which the emitted light remains polarized in a particular plane is proportional to the speed at which the molecules rotate and tumble in solution. Under constant pressure, temperature and viscosity, FP is directly related to the molecular weight of a fluorescent species. Therefore, when a small fluorescent molecule is incorporated into a larger molecule, there is an increase in FP. FP can be used in for genotyping of polymorphisms of interest (Chen et al, Genome Res, 1999; 9: 492-8 and Latif et al, Genome Res, 2001; 11; 436-40). FP can be utilized in 5′ nuclease assay (as described above), where the oligonucleotide probe is digested to a lower molecule weight species, for example is amenable to analysis by FP, but with the added benefit of not requiring a quencher. For example, Perlkin-Elmers AcycloPrime™-FP SNP Detection Kit can be used as a FP minisequencing method. Following PCR amplification, unicoportated primers and nucleotides are degraded enzymatically, the enzymes heat inactivated and a miniseqencing reaction using DNA polymerase and fluorescent-labelled dideoxynucleotides performed. FP is then measured, typically in a 96- to 386-well plate format on a FP-plate reader.

In some embodiments, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the individual, and incubated with the enzymes, DNA polymerase, ATP sulfurylase, luciferase and apyrase, and the substrates, adenosine 5′ phosphosulfate (APS) and luciferin. One of four deoxynucleotide triphosphates (dNTP), for example, corresponding to the nucleotide present in the mutation or polymorphism, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated and allows a clear determination of the presence or absence of, for example, the mutation or polymorphism. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATPS) is used as a substitute for the natural deoxyadenosine triphosphate (dATP) since it is efficiently used by the DNA polymerase, but not recognized by the luciferase. For detailed information about reaction conditions for the PYROSEQUENCING, see, e.g. U.S. Pat. No. 6,210,891, which is incorporated herein by reference in its entirety.

Other techniques known to persons skilled in the art are also incorporated for use with the present invention, for example see Kwok, Hum Mut 2002; 9; 315-323 and Kwok, Annu Rev Genomic Hum Genetics, 2001; 2; 235-58 for reviews, which are incorporated herein in their entirety by reference. Examples of other techniques to detect variances and/or polymorphisms are the INVADER® Assay (Gut et al, Hum Mutat, 2001; 17:475-92, Shi et al, Clin Chem, 2001, 47,164-92, and Olivier et al, Mutat Res, 2005; 573:103-110), the method utilizing FLAP endonucleases (U.S. Pat. No. 6,706,476) and the SNPlex genoptyping systems (Tobler et al, J. Biomol Tech, 2005; 16; 398-406.

In one embodiment, a long-range PCR (LR-PCR) is used to detect mutations or polymorphisms of the present invention. LR-PCR products are genotyped for mutations or polymorphisms using any genotyping methods known to one skilled in the art, and haplotypes inferred using mathematical approaches (e.g., Clark's algorithm (Clark (1990) Mol. Biol. Evol. 7:111-122).

For example, methods including complementary DNA (cDNA) arrays (Shalon et al., Genome Research 6(7):639-45, 1996; Bernard et al., Nucleic Acids Research 24(8):1435-42, 1996), solid-phase mini-sequencing technique (U.S. Pat. No. 6,013,431, Suomalainen et al. Mol. Biotechnol. June; 15(2):123-31, 2000), ion-pair high-performance liquid chromatography (Doris et al. J. Chromatogr. A can 8; 806(1):47-60, 1998), and 5′ nuclease assay or real-time RT-PCR (Holland et al. Proc Natl Acad Sci USA 88: 7276-7280, 1991), or primer extension methods described in the U.S. Pat. No. 6,355,433, can be used.

Molecular beacons also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity. Therefore, for example, two different molecular beacons are designed, one recognizing the mutation or polymorphism and the other the corresponding wildtype allele. When the molecular beacons hybridize to the nucleic acids, the fluorescent dye and quencher are separated, FRET does not occur, and the fluorescent dye emits light upon irradiation. Unlike TaqMan probes, molecular beacons are designed to remain intact during the amplification reaction, and must rebind to target in every cycle for signal measurement. TaqMan probes and molecular beacons allow multiple DNA species to be measured in the same sample (multiplex PCR), since fluorescent dyes with different emission spectra can be attached to the different probes, e.g. different dyes are used in making the probes for different disease-causing and SNP alleles. Multiplex PCR also allows internal controls to be co-amplified and permits allele discrimination in single-tube assays. (Ambion Inc, Austin, Tex., TechNotes 8(1)—February 2001, Real-time PCR goes prime time).

Another method to detect mutations or polymorphisms is by using fluorescence tagged dNTP/ddNTPs. In addition to use of the fluorescent label in the solid phase mini-sequencing method, a standard nucleic acid sequencing gel can be used to detect the fluorescent label incorporated into the PCR amplification product. A sequencing primer is designed to anneal next to the base differentiating the disease-causing and normal allele or the selected SNP alleles. A primer extension reaction is performed using chain terminating dideoxyribonucleoside triphosphates (ddNTPs) labeled with a fluorescent dye, one label attached to the ddNTP to be added to the standard nucleic acid and another to the ddNTP to be added to the target nucleic acid.

Alternatively, an INVADER® assay can be used (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535). For example, an INVADER® operating system (OS), provides a method for detecting and quantifying DNA and RNA. The INVADER® OS is based on a “perfect match” enzyme-substrate reaction. The INVADER® OS uses proprietary CLEAVASE® enzymes (Third Wave Technologies, Inc (Madison, Wis.)), which recognize and cut only the specific structure formed during the INVADER® process which structure differs between the different alleles selected for detection, i.e. the disease-causing allele and the normal allele as well as between the different selected SNPs. Unlike the PCR-based methods, the INVADER® OS relies on linear amplification of the signal generated by the INVADER® process, rather than on exponential amplification of the target.

In the INVADER® process, two short DNA probes hybridize to the target to form a structure recognized by the CLEAVASE® enzyme. The enzyme then cuts one of the probes to release a short DNA “flap.” Each released flap binds to a fluorescently-labeled probe and forms another cleavage structure. When the CLEAVASE® enzyme cuts the labeled probe, the probe emits a detectable fluorescence signal.

Mutations or polymophisms can also be detected using allele-specific hybridization followed by a MALDI-TOF-MS detection of the different hybridization products. In the preferred embodiment, the detection of the enhanced or amplified nucleic acids representing the different alleles is performed using matrix-assisted laser desorption ionization/time-of-flight (MALDI-TOF) mass spectrometric (MS) analysis described in the Examples below. This method differentiates the alleles based on their different mass and can be applied to analyze the products from the various above-described primer-extension methods or the INVADER® process.

In one embodiment, a haplotyping method can be used for the purpose of the invention. A halotyping method is a physical separation of alleles by cloning, followed by sequencing. Other methods of haplotyping include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotying method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N.J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

Other methods for genetic screening can be used within the scope of the present invention, for example, to detect mutations in genomic DNA, cDNA and/or RNA samples. Methods commonly used, or newly developed or methods yet unknown are encompassed for used in the present invention. Examples of newly discovered methods include for example, but are not limited to; SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471) and enzymatic mutation detection methods (Yeung et al, Biotechniques, 2005; 38; 749-758).

Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

In such embodiments, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e. g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

U.S. Pat. No. 4,946,773 describes an RNaseA mismatch cleavage assay that involves annealing single-stranded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches.

In other embodiments, alterations in electrophoretic mobility can be used to identify the particular allelic variant. For example, single strand conformation polymorphism (SSCP) can be used to detect differences in electrophoretic mobility between mutant and wild type nucleic acids (Orita et al. (1989) Proc Natl. Acad. Sol USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

Gel Migration Single strand conformational polymorphism (SSCP; M. Orita et al., Genomics 5:8 74-8 79 (1989); Huinphfies et al., In: Molecular Diagnosis of Genetic Diseases, R. Elles, ed. pp 321-340 (1996)) and temperature gradient gel electrophoresis (TGGE; R. M. Wartell et al., Nucl. Acids Res. 18:2699-2706 (1990)) are examples of suitable gel migration-based methods for determining the identity of a polymorphic site. In SSCP, a single strand of DNA will adopt a conformation that is uniquely dependent of its sequence composition. This conformation is usually different, if even a single base is changed. Thus, certain embodiments of the present invention, SSCP can be utilized to identify polymorphic sites, as wherein amplified products (or restriction fragments thereof of the target polynucleotide are denatured, then run on a non-denaturing gel. Alterations in the mobility of the resultant products are thus indicative of a base change. Suitable controls and knowledge of the “normal” migration patterns of the wild-type alleles can be used to identify polymorphic variants.

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for, example by adding a GC clamp of approximately 40 bp of high-melting GC rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Others have described using the MutS protein or other DNA-repair enzymes for detection of single-base mismatches. Alternative methods for detection of deletion, insertion or substitution mutations that can be used in the practice of the present invention are disclosed in U.S. Pat. Nos. 5,849,483, 5,851,770, 5,866,337, 5,925,525 and 5,928,870, each of which is incorporated herein by reference in its entirety. Several methods have been developed to screen polymorphisms and some examples are listed below. The reference of Kwok and Chen (2003) and Kwok (2001) provide overviews of some of these methods, both of these references are specifically incorporated by reference.

Examples of identifying polymorphisms and applying that information in a way that yields useful information regarding patients can be found, for example, in U.S. Pat. No. 6,472,157; U.S. Patent Application Publications 20020016293, 20030099960, 20040203034; WO 0180896, all of which are hereby incorporated by reference.

In another embodiment, multiplex PCR procedures using allele-specific primers can be used to simultaneously amplify multiple regions of a target nucleic acid (PCT Application WO89/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nad. Acad. Sci. (U.S.A) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT-Application WO89/06700; Kwoh, D. et al., Proc. Natl. Acad Sci. (U.S.A) 86:1173 Z1989); Gingeras, T. R. et al., PCT Application WO88/10315)), or isothermal amplification methods (Walker, G. T. et al., Proc. Natl. 4cad Sci. (U.S.A) 89:392-396 (1992)) can also be used.

Another method to determine genetic variation is using “gene chips.” Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

In some embodiments, the probe affixed to the surface of “gene chip” comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the probe affixed to the surface of “gene chip” comprises a nucleotide sequence selected from (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

Provided herein are methods, assays and systems for determining an increased risk for developing late onset AD in a subject by identifying the SNPs described herein or corresponding gene expression products in a biological sample of the subject. The term “biological sample” as used herein denotes a sample taken or isolated from a biological organism, e.g., tissue cell culture supernatant, cell lysate, a homogenate of a tissue sample from a subject or a fluid sample from a subject. Exemplary biological samples include, but are not limited to, blood, sputum, urine, spinal fluid, pleural fluid, nipple aspirates, lymph fluid, the external sections of the skin, respiratory, intestinal, and genitourinary tracts, tears, saliva, milk, feces, sperm, cells or cell cultures, serum, leukocyte fractions, smears, tissue samples of all kinds, embryos, etc. The term also includes both a mixture of the above-mentioned samples such as whole human blood containing mycobacteria as well as food samples that contain free or bound nucleic acids or cells containing nucleic acids. The term “biological sample” also includes untreated or pretreated (or pre-processed) biological samples.

A “biological sample” can contain cells from subject, but the term can also refer to non-cellular biological material, such as non-cellular fractions of blood, saliva, or urine, that can be used to measure gene expression levels or determine SNPs. In some embodiments, the sample is from a resection, biopsy, or core needle biopsy. In addition, fine needle aspirate samples can be used. Samples can be either paraffin-embedded or frozen tissue.

The sample can be obtained by removing a sample of cells from a subject, but can also be accomplished by using previously isolated cells (e.g. isolated by another person). In addition, the biological sample can be freshly collected or a previously collected sample. Furthermore, the biological sample can be utilized for the detection of the presence and/or quantitative level of a biomolecule of interest. Representative biomolecules include, but are not limited to, DNA, RNA, mRNA, polypeptides, and derivatives and fragments thereof. In some embodiments, the biological sample can be used for SNP determination for diagnosis of a disease or a disorder, e.g., Alzheimer's disease, using the methods, assays and systems of the invention.

In some embodiments, biological sample can be a biological fluid. Examples of biological fluids include, but are not limited to, saliva, bone marrow, blood, serum, plasma, urine, sputum, cerebrospinal fluid, an aspirate, tears, and any combinations thereof.

In some embodiments, the biological sample is an untreated biological sample. As used herein, the phrase “untreated biological sample” refers to a biological sample that has not had any prior sample pre-treatment except for dilution and/or suspension in a solution. Exemplary methods for treating a biological sample include, but are not limited to, centrifugation, filtration, sonication, homogenization, heating, freezing and thawing, and any combinations thereof.

In some embodiments, the biological sample is a frozen biological sample, e.g., a frozen tissue or fluid sample such as urine, blood, serum or plasma. The frozen sample can be thawed before employing methods, assays and systems of the invention. After thawing, a frozen sample can be centrifuged before being subjected to methods, assays and systems of the invention.

In some embodiments, the biological fluid sample can be treated with at least one chemical reagent, such as a protease inhibitor. In some embodiments, the biological fluid sample is a clarified biological fluid sample, for example, by centrifugation and collection of a supernatant comprising the clarified biological fluid sample.

In some embodiments, a biological sample is a pre-processed biological sample, for example, supernatant or filtrate resulting from a treatment selected from the group consisting of centrifugation, filtration, sonication, homogenization, lysis, thawing, amplification, purification, restriction enzyme digestion ligation and any combinations thereof. In some embodiments, a biological sample can be a nucleic acid product amplified after polymerase chain reaction (PCR). The term “nucleic acid” used herein refers to DNA, RNA, or mRNA.

In some embodiments, the biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

The skilled artisan is well aware of methods and processes appropriate for pre-processing of biological samples required for determination of SNPs or expression of gene expression products as described herein.

The methods and assay disclosed herein can be carried out in an automated and/or high-throughput system. Accordingly, in one aspect, the disclosure provides a computer system comprising: (a) a determination module configured to identify and detect at least one single nucleotide polymorphism (SNP) in a biological sample of a subject, wherein the SNP is selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO: 1 is a portion of genomic nucleic acid sequence of PLXNA4; and (iv) any combinations thereof (b) a storage module configured to store output data from the determination module; (c) a computing module adapted to identify from the output data at least one of AD risk associated alleles is present in the output data stored on the storage module; and (d) a display module for displaying if any of the AD risk associated alleles was identified or not, and/or displaying the detected alleles.

In some embodiments, the computer system can include: (a) at least one memory containing at least one computer program adapted to control the operation of the computer system to implement a method that includes: (i) receiving data of the level of expression or intensity of signal of measured for a PLXNA4 isoform (e.g. TS1 or TS3) mRNA; (ii) generating a report of intensity of expression or intensity of signal of measured PLXNA4 isoform mRNA in a biological sample and optionally a reference level for PLXNA4 isoform mRNA signal intensity; and (b) at least one processor for executing the computer program.

In some embodiments, a computer system can include: (a) at least one memory containing at least one computer program adapted to control the operation of the computer system to implement a method that includes: (i) receiving data of the level of expression or intensity of signal of measured AD risk associated allele levels (ii) generating a report of intensity of expression or intensity of signal of measured AD risk associated allele levels in a biological sample and optionally a reference level AD risk associated allele signal intensity; and (b) at least one processor for executing the computer program.

In some embodiments, a computer system can include, for example, an Intel or AMD x86 based single or multi-core central processing unit (CPU), an ARM processor or similar computer processor for processing the data. The CPU or microprocessor can be any conventional general purpose single- or multi-chip microprocessor such as an Intel and AMD processor, a SPARC processor, or an ARM processor. In addition, the microprocessor may be any conventional or special purpose microprocessor such as a digital signal processor or a graphics processor. The microprocessor typically has conventional address lines, conventional data lines, and one or more conventional control lines. As described below, the software according to the invention can be executed on dedicated system or on a general purpose computer having a DOS, CPM, Windows, Unix, Linix or other operating system. The system can include non-volatile memory, such as disk memory and solid state memory for storing computer programs, software and data and volatile memory, such as high speed ram for executing programs and software.

Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media can be any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks), Blue-ray, USB drives, micro-SD drives, or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing.

The present invention can be implemented on a stand-alone computer or as part of a networked computer system. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices. In one embodiment, the invention can use a client-server environment over a network, e.g., a public network such as the internet or a private network to connect to data and resources stored in remote and/or centrally located locations. In this embodiment, a server such as a web server can provide access, e.g. open access, pay as you go or subscription based access, to the information provided according to the invention. In a client server environment, a client computer executing a client software or program, such as a web browser, connects to the server over the network. The client software provides a user interface for a user of the invention to input data and information and receive access to data and information. The client software can be viewed on a local computer display or other output device and can allow the user to input information, such as by using a computer keyboard, mouse or other input device. The server executes one or more computer programs that receives data input through the client software, processes data according to the invention and outputs data to the user, as well as provide access to local and remote computer resources. For example, the user interface can include a graphical user interface comprising an access element, such as a text box, that permits entry of data from the assay, e.g., the data from a positive reference cancer cell, as well as a display element that can provide a graphical read out of the results of a comparison with a cancer cell with a known metastatic potential or invasive capacity, or data sets transmitted to or made available by a processor following execution of the instructions encoded on a computer-readable medium.

Embodiments of the invention also provide for systems (and computer readable medium providing instructions for causing computer systems) to perform a method for determining quality assurance of a pluripotent stem cell population according to the methods as disclosed herein.

In some embodiments of the invention, the computer system software can include one or more functional modules, which can be defined by computer executable instructions recorded on computer readable media and which cause a computer to perform, when executed, a method according to one or more embodiments of the invention. The modules can be segregated by function for the sake of clarity, however, it should be understood that the modules need not correspond to discreet blocks of code and the described functions can be carried out by the execution of various software code portions stored on various media and executed at various times. Furthermore, it should be appreciated that the modules can perform other functions, thus the modules are not limited to having any particular function or set of functions. In some embodiments, functional modules are, for example, but are not limited to, an array module, a determination module, a storage module, a reference comparison module, a normalization module, and a display module to display the results (e.g., the invasive potential of the test cancer cell population). The functional modules can be executed using one or multiple computers, and by using one or multiple computer networks.

The information embodied on one or more computer-readable media can include data, computer software or programs, and program instructions, which as a result of being executed by a computer, transform the computer to special purpose machine and can cause the computer to perform one or more of the functions described herein. Such instructions can be originally written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied can reside on one or more of the components of a computer system or a network of computer systems according to the invention.

In some embodiments, a computer-readable media can be transportable such that the instructions stored thereon can be loaded onto any computer resource to implement the aspects of the present invention discussed herein. In addition, it should be appreciated that the instructions stored on computer readable media are not limited to instructions embodied as part of an application program running on a host computer. Rather, the instructions may be embodied as any type of computer code (e.g., object code, software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

Where the quantity to be measured is protein expression, the system as disclosed herein can be configured to receive data from an automated protein analysis systems, for example, using immunoassay, for example western blot analysis or ELISA, or a high through-put protein detection method, for example but are not limited to automated immunohistochemistry apparatus, for example, robotically automated immunodetection apparatus which in an automated system can perform immunohistochemistry procedure and detect intensity of immunostaining, such as intensity of an antibody staining of the substrates and produce output data. Examples of such automated immunohistochemistry apparatus are commercially available, and can be readily adapted to automatically detect the level of protein expression in the assay as disclosed herein, and include, for example but not limited to such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

In some embodiments, a system as disclosed herein, can receive data of intensity of protein expression of PLXNA4 from an automated ELISA system (e.g. DSX® or DK® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters), or adapted systems thereof for detecting cells on the separated substrates as disclosed herein.

In some embodiments, a system as disclosed herein, can receive data can receive data of intensity of mRNA expression of PLXNA4 (e.g., isoform TS1 or TS3) or presence of absence of an AD risk associated allele from any method of determining gene or nucleic acid expression or mutations or SNP genotyping. In some embodiments, the system as disclosed herein can be configured to receive data from an automated gene expression analysis system, e.g., an automated protein expression analysis including but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array.

In some embodiments of the present invention, an automated gene expression analysis system can record the data electronically or digitally, annotated and retrieved from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

In some embodiments, a system as disclosed herein, can receive data from an allele-specific PCR. The term “allele-specific PCR” refers to PCR techniques where the primer pairs are chosen such that amplification is dependent upon the input template nucleic acid containing the polymorphism of interest. In such embodiments, primer pairs are chosen such that at least one primer is an allele-specific oligonucleotide primer. In some sub-embodiments of the present invention, allele-specific primers are chosen so that amplification creates a restriction site, facilitating identification of a polymorphic site. In other embodiments of the present invention, amplification of the target polynucleotide is by multiplex PCR (Wallace et al. (PCT Application WO89/10414)). Through the use of multiplex PCR, a multiplicity of regions of a target polynucleotide can be amplified simultaneously. This is particularly advantageous in embodiments where more than one SNP is to be detected.

In another embodiment, multiplex PCR procedures using allele-specific primers can be used to simultaneously amplify multiple regions of a target nucleic acid (PCT Application WO89/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nad. Acad. Sci. (U.S.A) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT-Application WO89/06700; Kwoh, D. et al., Proc. Natl. Acad Sci. (U.S.A) 86:1173 Z1989); Gingeras, T. R. et al., PCT Application WO88/10315)), or isothermal amplification methods (Walker, G. T. et al., Proc. Natl. 4cad Sci. (U.S.A) 89:392-396 (1992)) can also be used.

In some embodiments, a system as disclosed herein, can receive data from any genotyping assay known by persons of ordinary skill in the art, including, but not limited to, those disclosed in U.S. Pat. No. 6,472,157; U.S. Patent Application Publications 20020016293, 20030099960, 20040203034; WO 0180896, all of which are hereby incorporated by reference, or by linkage disequlibrium, restriction fragment length polymorphism” (RFLP) analysis, single strand conformational polymorphism (SSCP), RNaseI for mismatch detection, SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471), enzymatic mutation detection methods (Yeung et al, Biotechniques, 2005; 38; 749-758), matrix-assisted laser desorption ionization/time-of-flight (MALDI-TOF) mass spectrometric (MS) analysis, long-range PCR (LR-PCR), genotype assays disclosed in Kwok, Hum Mut 2002; 9; 315-323 and Kwok, Annu Rev Genomic Hum Genetics, 2001; 2; 235-58, (which are incorporated herein in their entirety by reference), INVADER® Assay (Gut et al, Hum Mutat, 2001; 17:475-92, Shi et al, Clin Chem, 2001, 47,164-92, and Olivier et al, Mutat Res, 2005; 573:103-110), the method utilizing FLAP endonucleases (U.S. Pat. No. 6,706,476) and the SNPlex genoptyping systems (Tobler et al, J. Biomol Tech, 2005; 16; 398-406) and other such genotyping assays known to one of ordinary skill in the art.

In some embodiments, the data can be received from a memory, a storage device, or a database. The memory, storage device or database can be directly connected to the computer system retrieving the data, or connected to the computer through a wired or wireless connection technology and retrieved from a remote device or system over the wired or wireless connection. Further, the memory, storage device or database, can be located remotely from the computer system from which it is retrieved.

Examples of suitable connection technologies for use with the present invention include, for example parallel interfaces (e.g., PATA), serial interfaces (e.g., SATA, USB, Firewire, local area networks (LAN), wide area networks (WAN), Internet, Intranet, and Extranet, and wireless (e.g., Blue Tooth, Zigbee, WiFi, WiMAX, 3G, 4G) communication technologies

Storage devices are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytosine level information. Such information can be provided in digital form that can be transmitted and read electronically, e.g., via the Internet, on diskette, via USB (universal serial bus) or via any other suitable mode of communication.

As used herein, “stored” refers to a process for recording information, e.g., data, programs and instructions, on the storage device that can be read back at a later time. Those skilled in the art can readily adopt any of the presently known methods for recording information on known media to contribute to the data of (i) the level of expression of a PLXNA4 isoform (TS1 or TS3) mRNA and/or (ii) presence or absence of an AD risk associated allele as disclosed in the methods herein.

A variety of software programs and formats can be used to store information on the storage device. Any number of data processor structuring formats (e.g., text file or database) can be employed to obtain or create a medium having recorded scorecard thereon.

In some embodiment, the system has a processor for running one or more programs, e.g., where the programs can include an operating system (e.g., UNIX, Windows), a relational database management system, an application program, and a World Wide Web server program. The application program can be a World Wide Web application that includes the executable code necessary for generation of database language statements (e.g., Structured Query Language (SQL) statements). The executable can include embedded SQL statements. In addition, the World Wide Web application can include a configuration file which contains pointers and addresses to the various software entities that provide the World Wide Web server functions as well as the various external and internal databases which can be accessed to service user requests. The Configuration file can also direct requests for server resources to the appropriate hardware devices, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the system as disclosed herein can be used to compare the data of intensity of one or more of (i) the level of expression of PLXNA4 mRNA (e.g. isoform TS1 or TS3); and (ii) presence of an AD risk associated allele; and generate a report of the presence or absence, or amount of (i) the expression of the PLXNA4 mRNA; (ii) the AD risk associated allele with reference data (e.g., reference intensity values), as disclosed herein.

In some embodiments of this aspect and all other aspects of the present invention, the system can compare the data in a “comparison module” which can use a variety of available software programs and formats for the comparison operative to compare sequence information determined in the determination module to reference data. In one embodiment, the comparison module is configured to use pattern recognition techniques to compare levels of expression (e.g., mRNA levels and/or protein levels) as well as compare sequence information (e.g., identify the presence of different SNPs of AD risk associated alleles from one or more entries to one or more reference data patterns. The comparison module can be configured using existing commercially-available or freely-available software for comparing patterns, and may be optimized for particular data comparisons that are conducted. The comparison module can also provide computer readable information related to the level or amount of intensity of expression of the level of expression of a specific PLXNA4 isoform (e.g., TS1 or TS3) mRNA.

By providing data of the intensity of expression of (i) the level of expression of a PLXN4 isoform mRNA; and/or (ii) presence of an AD risk associated allele, in computer-readable form, one can use the data to compare with data within the storage device. For example, search programs can be used to identify relevant reference that match the same pattern. The comparison made in computer-readable form provides computer readable content which can be processed by a variety of means. The content can be retrieved from the comparison module, the retrieved content.

In some embodiments, the comparison module provides computer readable comparison result that can be processed in computer readable form by predefined criteria, or criteria defined by a user, to provide a report which comprises content based in part on the comparison result that may be stored and output as requested by a user using a display module. In some embodiments, a display module enables display of a content based in part on the comparison result for the user, wherein the content is a report indicative of the results of the comparison of the intensity of expression of (i) the level of expression of a PLXNA4 isoform mRNA; and/or (ii) presence of an AD risk associated allele with their respective reference values.

In some embodiments, the display module enables display of a report or content based in part on the comparison result for the end user, wherein the content is a report indicative of the results of the comparison of the intensity of expression of any one or more of (i) the level of expression of PLXNA4 isoform (e.g., TS1 or TS2) mRNA; and/or (ii) presence of an AD risk associated allele.

In some embodiments, the comparison module, or any other module of the invention, can include an operating system (e.g., UNIX, Windows) on which runs a relational database management system, a World Wide Web application, and a World Wide Web server. World Wide Web application can includes the executable code necessary for generation of database language statements [e.g., Standard Query Language (SQL) statements]. The executables can include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using an HTML interface provided by Web browsers and Web servers. In other embodiments of the invention, other interfaces, such as HTTP, FTP, SSH and VPN based interfaces can be used to connect to the Internet databases.

In some embodiments, a computer-readable media can be transportable such that the instructions stored thereon, such as computer programs and software, can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions can be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The computer instructions can be implemented in software, firmware or hardware and include any type of programmed step undertaken by modules of the information processing system. The computer system can be connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

In some embodiments, the computer system as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). The computing devices can be desktop devices, servers, portable computers, hand-held computing devices, smart phones, set-top devices, or any other desired type or configuration. As used herein, a network includes one or more of the following, including a public internet, a private internet, a secure internet, a private network, a public network, a value-added network, an intranet, an extranet and combinations of the foregoing.

In some embodiments, a comparison module provides computer readable data that can be processed in computer readable form by predefined criteria, or criteria defined by a user, to provide a retrieved content that may be stored and output as requested by a user using a display module.

In some embodiments, the computerized system can include or be operatively connected to an output module. In some embodiments, the output module is a display module, such as computer monitor, touch screen or video display system. The display module allows user instructions to be presented to the user of the system, to view inputs to the system and for the system to display the results to the user as part of a user interface. Optionally, the computerized system can include or be operative connected to a printing device for producing printed copies of information output by the system.

In some embodiments, the results can be displayed on a display module or printed in a report, e.g., a to indicate any one or more of (i) the level of expression of a PLXNA4 isoform (e.g., TS1 or TS3) mRNA; and/or (ii) presence of at least one AD risk associated allele. In some embodiments, the report is a hard copy printed from a printer.

In alternative embodiments, the computerized system can use light or sound to report the result. For example, in all aspects of the invention, the report produced by the methods, assays, systems and kits as disclosed herein can comprise a report which is color coded to signal or indicate any one or more of (i) the level of expression of a PLXNA4 (eg., isoform TS1 or TS3) mRNA; and/or (ii) presence of at least one AD risk associated allele.

In some embodiments, the report can also present text, either verbally or written, giving a recommendation of if a subject is amenable to treatment with a TS1 PLXNA4 inhibitory agent as disclosed herein. In other embodiments, the report provides just values or numerical scores for the presence of any one or more of the (i) the level of expression of PLXNA4 mRNA; and/or (ii) presence of at least one AD risk associated allele which can be readily compared by a physician with reference values as disclosed herein.

In some embodiments, the report data from the comparison module can be displayed on a computer monitor as one or more pages of the printed report. In one embodiment of the invention, a page of the retrieved content can be displayed through printable media. The display module can be any device or system adapted for display of computer readable information to a user. The display module can include speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc.

In some embodiments, a World Wide Web browser can be used to provide a user interface to allow the user to interact with the system to input information, construct requests and to display retrieved content. In addition, the various functional modules of the system can be adapted to use a web browser to provide a user interface. Using a Web browser, a user can construct requests for retrieving data from data sources, such as data bases and interact with the comparison module to perform comparisons and pattern matching. The user can point to and click on user interface elements such as buttons, pull down menus, scroll bars, etc. conventionally employed in graphical user interfaces to interact with the system and cause the system to perform the methods of the invention. The requests formulated with the user's Web browser can be transmitted over a network to a Web application that can process or format the request to produce a query of one or more database that can be employed to provide the pertinent information.

The present disclosure further provides methods for treating AD in a subject. In accordance with embodiments of the various aspects disclosed herein, PLXNA4 is a viable target for therapeutic treatment of AD. Accordingly, provided herein is a method for treatment of AD in a subject. Generally the method comprises administering to the subject a pharmaceutically acceptable composition comprising a TS1 PLXNA4 inhibitory agent.

Subjects amenable to methods of treatment are subjects that have been diagnosed with Alzheimer's disease. Methods for diagnosing Alzheimer's disease are well known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in U.S. Pat. No. 7,771,937, U.S. Pat. No. 7,595,167, US 55580748, and PCT Application No.: WO2009/009457, the content of which is incorporated by reference in its entirety. Additionally, protein-based biomarkers for AD, some of which can be detected by non-invasive imaging, e.g., PET, are disclosed in U.S. Pat. No. 7,794,948, the content of which is incorporated by reference in its entirety.

Genes involved in AD risk can be used for diagnosis of AD, including the SNPs described herein. Accordingly, in one embodiment, the methods provided herein for identifying a nucleic acid polymorphism in a biological sample can also be used for screening AD in a subject. Such AD “risk genes” increase the risk of developing AD. In addition, one example of other AD risk genes is apolipoprotein E-ε4 (APOE-ε4). APOE-ε4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-ε4 have an increased risk of developing AD. Those who inherit two copies have an even higher risk, but not a certainty of developing AD. In addition to raising risk, APOE-ε4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference in its entirety. Genetic tests are well established in the art and are available, for example for APOE-e4. A subject carrying the APOE-ε4 allele can, therefore, be identified as a subject at risk of developing AD.

In further embodiments, subjects with Aβ burden are amenable to the methods described herein. Such subjects include, but not limited to, the ones with Down syndrome, Huntington disease, the unaffected carriers of APP or presenilin gene mutations, and the late onset AD risk factor, apolipoprotein E-ε4.

In some embodiments, AD patients that are currently receiving other AD therapeutic treatment can also be subjected to the methods of treatment as described herein.

In some embodiments, a subject who has been diagnosed with an increased risk for developing AD, e.g., using the diagnostic methods and assays described herein or any AD diagnostic methods known in the art, can be subjected to the methods of treatment as described herein.

In some embodiments, the subject with at least one AD risk-associated allele but no AD symptoms, including undetectable level of amyloid-beta protein in the brain and/or no detectable cognitive impairment can be administered with a preventive treatment. These treatment of prevention interventions include, but are not limited to life style advice, including e.g., prescribing an aerobic exercise regime to exercise the body and/or mental exercises to keep brain active, dietary advice, including increase in intake of omega-3 fatty acids, fruits and vegetables, fish or poultry, whole-grain breads and cereals, or reduction of sugar or cholesterol rich food intake to lower cholesterol, and administering pharmaceutical agents effective in prevention or treatment of AD.

In some embodiments, the subject having at least one AD risk-associated allele and exhibiting AD symptoms, i.e. diagnosed with AD, can be treated with the methods of treatment described herein. In some embodiments, the subject diagnosed with AD can be treated with a drug known in the art such as cholinesterase inhibitors (for example, ARICEPT®), the glutamate antagonist NAMENDA® and dimebolin, which is currently in clinical trials. The subject diagnosed with AD can further be advised on changes in life style and/or diet to slow down the progression of AD. Accordingly, the term “treatment or prevention for AD” will encompass treating a subject diagnosed with AD to slow down or ameliorate at least one symptom associated with AD, or treating a subject with an increased risk for AD, e.g, carrying an AD risk associated allele described herein to avoid or delay the onset of AD. The term “prevention” as used herein refers to a complete avoidance of symptoms, such as cognitive impairment or measurable markers of AD, level of Aβ in the brain, or delay the onset of AD. Inhibition of AD development is also considered a preventive measure even if it does not confer a complete avoidance of AD symptoms. The term “inhibition” as used in reference to the development of a disease (e.g., AD) refer to a reduced severity or degree of any one or more of those symptoms or markers, relative to those symptoms or markers arising in a control or non-treated individual with a similar likelihood or susceptibility of developing AD, or relative to symptoms or markers likely to arise based on historical or statistical measures of populations affected by AD. By “reduced severity” is meant at least about 20% in the severity or degree of a symptom or measurable marker, e.g., level of Aβ in the brain, relative to a control, such as without administration of the treatment described herein, e.g., at least about 20%, 30%, 40%, 50%, 60%, 70%, 80%, 90%, 95%, 99% or even 100% (i.e., no or non-detectable level of cognitive impairment or measurable markers, e.g., Aβ level).

In another aspect, the disclosure provides kits for the practice of the assays and methods disclosed herein. The kits preferably include one or more containers containing a TS1 PLXNA4 inhibitory agent and a pharmaceutically acceptable excipient. The kit can optionally contain additional therapeutics to be co-administered with the TS1 PLXNA4 inhibitory agent. The kit can comprise instructions for administration of the TS1 PLXNA4 inhibitory agent to a subject with AD or at risk of AD.

The kits can also optionally include appropriate systems (e.g. opaque containers) or stabilizers (e.g. antioxidants) to prevent degradation of the TS1 PLXNA4 inhibitory agent by light or other adverse conditions.

Another aspect relates to kit to detect the presence of one or more of: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, in a subject.

In some embodiments, the kit can comprise probes, e.g., allele-specific oligonucleotide probes or allele specific primer probes for detecting the SNP1, SNP2, and/or SNP3 loci in a sample from a subject. In some embodiments, the kit can comprise probes, e.g., allele-specific oligonucleotide probes or allele specific primer probes for detecting one or more of: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, for the practice of the methods of this invention.

Allele-specific probes are well know by persons of ordinary skill in the art, with oligonucleotides encompassed for use as probes, and refer to such as genomic DNA, mRNA, or other suitable sources of nucleic acid oligonucleotides. For such purposes, the oligonucleotides must be capable of specifically hybridizing to a target polynucleotide or DNA nucleic acid molecule. As used herein, two nucleic acid molecules are said to be capable of specifically hybridizing to one another if the two molecules are capable of forming an anti-parallel, double-stranded nucleic acid structure under hybridizing conditions.

The term “allele-specific oligonucleotide” or “ASO” refers to an oligonucleotide that is able to hybridize to a region of a target polynucleotide spanning the sequence, mutation, or polymorphism being detected and is substantially unable to hybridize to a corresponding region of a target polynucleotide that either does not contain the sequence, mutation, or polymorphism being detected or contains an altered sequence, mutation, or polymorphism. As will be appreciated by those in the art, allele-specific is not meant to denote an absolute condition. Allele-specificity will depend upon a variety of environmental conditions, including salt and formamide concentrations, hybridization and washing conditions and stringency. Depending on the sequences being analyzed, one or more allele-specific oligonucleotides can be employed for each target polynucleotide. Preferably, allele-specific oligonucleotides will be completely complementary to the target polynucleotide. However, departures from complete complementarity are permissible. In order for an oligonucleotide to serve as a primer oligonucleotide, however, it typically need only be sufficiently complementary in sequence to be able to form a stable double-stranded structure under the particular environmental conditions employed. Establishing environmental conditions typically involves selection of solvent and salt concentration, incubation temperatures, and incubation times.

In some embodiments, the ASO comprises a nucleotide sequence substantially complementary to: (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the ASO comprises a nucleotide sequence selected from (i) GGTCCTCGCCTCC (SEQ ID NO: 2); (ii) GGTCCTAGCCTCC (SEQ ID NO: 3); (iii) GTTTGCCGTGTCG (SEQ ID NO: 4); (iv) GTTTGCTGTGTCG (SEQ ID NO: 5); (v) TCCCAAACTCCTG (SEQ ID NO: 6); (vi) TCCCAACCTCCTG (SEQ ID NO: 7); or (vii) any combinations of (i)-(vi).

In some embodiments, the kit can be used to perform a genotyping assay used to determine the AD risk associated SNPs disclosed herein, where the genotyping assay is selected from any or a combination in the group consisting of: PCR-based assays, RT-PCR, nucleic acid hybridization, sequence analysis, TaqMan SNP genotyping probes, microarrays, direct or indirect sequencing, restriction site analysis, hybridization based genotyping assays, gel migration assays, antibodies assays, fluorescent polarization, mass spectroscopy, allele-specific PCR, single-strand conformational polymorphism (SSCP) analysis, heteroduplex analysis, oligonucleotide ligation, PCR-RFLP, allele-specific amplification (ASA), single-molecule dilution (SMD), coupled amplification and sequencing (CAS), Restriction enzyme analysis, restriction fragment length polymorphism (RFLP), ligation based assays, single base extension (or minisequencing), MALDI-TOF, and homogenous assays.

The kits can optionally include instructional materials containing directions (i.e., protocols) providing for the use of a compounds and composition in the treatment of AD. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Kits for determining if a subject is at increased risk of developing Alzheimer's disease will include at least one reagent specific for detecting for the presence or absence of the AD risk associated SNPs described herein or antibodies specific for detecting the gene expression products (e.g., PLXNA4) associated with AD risk associated SNPs, and instructions for observing that the subject is at increased risk of developing Alzheimer's disease if the presence of at least one of the SNPs described herein is detected. The kit may optionally include a nucleic acid for detection of the gene of interest.

Diagnostic kits for carrying out antibody assays can be produced in a number of ways. In one embodiment, the diagnostic kit comprises (a) an antibody which binds PLXNA4 conjugated to a solid support and (b) a second antibody which binds PLXNA4 conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. A second embodiment of a test kit comprises (a) an antibody as above, and (b) a specific binding partner for the antibody conjugated to a detectable group. Ancillary agents as described above can likewise be included. The test kit may be packaged in any suitable manner, typically with all elements in a single container along with a sheet of printed instructions for carrying out the test. In other embodiments, the diagnostic kits can comprise primers or probes for detection of mRNA level of PLXNA4 isoform TS1 and/or TS3.

Exemplary embodiments of the various aspects disclosed herein can be described by one or more of the following numbered paragraphs:
1. A method for inhibiting progression of Alzheimer's disease, the method comprising administering to a subject having or at risk for Alzheimer's disease a therapeutically effective amount of a TS1 PLXNA4 inhibitory agent.
2. A method for inhibiting progression of Alzheimer's disease in a subject in need thereof, the method comprising administering to a subject determined to have one or more of AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO: 1 is a portion of genomic nucleic acid sequence of PLXNA4; and (iv) any combinations thereof, a therapeutically effective amount of a TS1 PLXNA4 inhibitory agent.
3. A method for inhibiting or reducing neurofibrillary tangles in the brain, the method comprising administering to a subject having or at risk for having neurofibrillary tangles in the brain a therapeutically effective amount of a TS1 PLXNA4 inhibitory agent.
4. A method for inhibiting or reducing neurofibrillary tangles in the brain of a subject in need thereof, the method comprising administering to a subject determined to have one or more of AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, a therapeutically effective amount of a TS1 PLXNA4 inhibitory agent.
5. A method for inhibiting or reducing tau phosphorylation in the brain, the method comprising administering to a subject in need thereof a therapeutically effective amount of a TS1 PLXNA4 inhibitory agent.
6. A method for inhibiting or reducing tau phosphorylation in the brain of a subject in need thereof, the method comprising administering to a subject determined to have one or more of AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, a therapeutically effective amount of a TS1 PLXNA4 inhibitory agent.
7. A method for treating a subject having or at risk for Alzheimer's disease, comprising administering a therapeutically effective amount of a TS1 PLXNA4 inhibitory agent to a subject in need thereof
8. A method for treating a subject having or at risk for Alzheimer's disease, the method comprising administering to a subject determined to have one or more of AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, a therapeutically effective amount of a TS1 PLXNA4 inhibitory agent.
9. The method of any of paragraphs 1-8, wherein the subject is determined to have two or more AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof
10. The method of any of paragraphs 1-9, wherein the subject is determined to have three AD risk associated single nucleotide polymorphism (SNP) selected from: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4.
11. The method any one of paragraphs 1-10, wherein the TS1 PLXNA4 inhibitory agent is selected from the group consisting of small molecules, nucleic acids, nucleic acid analogues, peptides, proteins, antibodies, antigen binding fragments of antibodies, and any combinations thereof
12. The method of any of paragraphs 1-11, wherein the TS1 PLXNA4 inhibitory agent is an oligonucleotide.
13. The method of any of paragraphs 1-12, wherein the TS1 PLXNA4 inhibitory agent is an anti-miR, antagomir, antisense oligonucleotide, ribozyme, aptamer, siRNA, shRNA, or RNAi agent.
14. The method of any one of paragraphs 1-13, wherein the TS1 PLXNA4 inhibitory agent does not bind or inhibit TS2 PLXNA4 or TS3 PLXNA4.
15. The method of any of paragraphs 1-14, further comprising a step of diagnosing the subject with AD or risk of AD prior to said administering.
16. The method of any of paragraphs 1-15, further comprising assaying a biological sample from the subject before onset of said administering, wherein said assaying comprising measuring the absence of presence of a SNP selected from the group consisting of: (i) SNP1 genotype A/A or A/C (or T/T or T/G in the complement) of SEQ ID NO: 1, wherein SNP1 is identified by rs277472 on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235, wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 on SEQ ID NO: 1, wherein the SEQ ID NO. 1 is a portion of genomic nucleic acid sequence of PLXNA4; and (iv) any combinations thereof, wherein presence of one or more of SNP1-SNP3 is indicative of proceeding with said administering regimen.
17. The method of paragraph 16, wherein said assaying comprises: subjecting the biological sample from a subject to at least one genotyping assay that determines the genotypes of at least one (e.g., one, two, or three) loci, wherein said loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof
18. The method of paragraph 16 or 17, wherein said assaying comprises:
   a. contacting the biological sample with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. washing the sample to remove unbound oligonucleotide;
   c. measuring the intensity of the signal from the bound, detectable bound detectable oligonucleotide;
   d. comparing the measured intensity of the signal with a reference value, wherein an increased measured intensity relative to the reference value is indicative of presence of at least one of SNP1-SNP3.
19. The method of any of paragraphs 1-18, further comprising assaying a biological sample from the subject before onset of said administering, wherein said assaying comprising measuring the expression or level of TS1 or TS3 PLXNA4, wherein an increased level of expression or amount of TS1 or TS3 PLXNA4, is indicative of proceeding with said administering regimen.
20. The method of paragraph 19, where said assaying comprises:
   a. contacting a biological sample obtained from a subject with a detectable antibody specific for TS1 or TS3 PLXNA4 or detectable nucleic acid for TS1 or TS3 PLXNA4;
   b. washing the sample to remove unbound antibody or unbound nucleic acid;
   c. measuring the intensity of the signal from the bound, detectable antibody or bound detectable nucleic acid;
   d. comparing the measured intensity of the signal with a reference value and if the measured intensity is increased relative to the reference value; and
   e. identifying the subject as having an increased probability of having AD.
21. An assay comprising:
   a. subjecting a test sample from a subject to at least one genotyping assay that determines the genotypes of at least one (e.g., one, two, or three) loci, wherein said loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof; and
   b. identifying the subject as having an increased probability of having AD when at least one of the following combinations of SNPs is determined to be present: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof
22. The assay of paragraph 21, wherein said loci of step (a) are further selected from: (i) SNP4, wherein SNP4 is identified by rs1593222 of SEQ ID NO: 1, wherein the SEQ ID NO. 1 is a portion of genomic nucleic acid sequence of PLXNA4; (ii) SNP5, wherein SNP5 is identified by rs6959579 of SEQ ID NO: 1, wherein the SEQ ID NO. 1 is a portion of genomic nucleic acid sequence of PLXNA4; (iii) SNP6, wherein SNP6 is identified by rs17166339 of SEQ ID NO: 1, wherein the SEQ ID NO. 1 is a portion of genomic nucleic acid sequence of PLXNA4.
23. An assay comprising:
   a. transforming at least one nucleic acid polymorphism in a locus in a biological sample from the subject into at least one detectable target, wherein the locus is selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof and
   b. detecting presence or absence of at least one AD risk associated SNP from the at least one detectable target, wherein the at least one AD risk associated SNP is selected from: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof and
   c. identifying the subject as having an increased probability of having AD if presence of at least one AD risk associated SNP is detected.
24. An assay comprising:
   a. contacting a biological sample obtained from a subject with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. washing the sample to remove unbound oligonucleotide;
   c. measuring the intensity of the signal from the bound, detectable bound detectable oligonucleotide;
   d. comparing the measured intensity of the signal with a reference value and if the measured intensity is increased relative to the reference value; and
   e. identifying the subject as having an increased probability of having AD.
25. An assay for identifying a subject having or at risk for Alzheimer's disease comprising:
   a. subjecting a test sample from a subject to at least one genotyping assay that determines the genotypes of at least one (e.g., one, two, or three) loci, wherein said loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof; and
   b. identifying the subject as having an increased probability of having AD when at least one of the following combinations of SNPs is determined to be present: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof
26. An assay for identifying a subject having or at risk for Alzheimer's disease comprising:

a. transforming at least one nucleic acid polymorphism in a locus in a biological sample from the subject into at least one detectable target, wherein the locus is selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof; and
b. detecting presence or absence of at least one AD risk associated SNP from the at least one detectable target, wherein the at least one AD risk associated SNP is selected from: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof; and
c. identifying the subject as having an increased probability of having AD when at least one AD risk associated SNP is detected in step (b).
27. An assay for identifying a subject having or at risk for Alzheimer's disease comprising:
   a. contacting a biological sample obtained from a subject with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. washing the sample to remove unbound oligonucleotide;
   c. measuring the intensity of the signal from the bound, detectable bound detectable oligonucleotide;
   d. comparing the measured intensity of the signal with a reference value and if the measured intensity is increased relative to the reference value; and
   e. identifying the subject as having an increased probability of having AD.
28. An assay for determining if a subject is in need of treatment or prevention for Alzheimer's disease, comprising:
   a. subjecting a test sample from a subject to at least one genotyping assay that determines the genotypes of at least one (e.g., one, two, or three) loci, wherein said loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof and
   b. identifying or selecting the subject for treatment or prevention for AD when at least one of the following combinations of SNPs is determined to be present: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof
29. An assay for determining if a subject is in need of treatment or prevention for Alzheimer's disease, comprising:
   a. transforming at least one nucleic acid polymorphism in a locus in a biological sample from the subject into at least one detectable target, wherein the locus is selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof and
   b. detecting presence or absence of at least one AD risk associated SNP from the at least one detectable target, wherein the at least one AD risk associated SNP is selected from: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof; and
   c. identifying or selecting the subject for treatment or prevention for AD when at least one AD risk associated SNP is detected in step (b).
30. An assay for determining if a subject is in need of treatment or prevention for Alzheimer's disease, comprising:
   a. contacting a biological sample obtained from a subject with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. washing the sample to remove unbound oligonucleotide;
   c. measuring the intensity of the signal from the bound, detectable bound detectable oligonucleotide;
   d. comparing the measured intensity of the signal with a reference value and if the measured intensity is increased relative to the reference value; and
   e. identifying or selecting the subject for treatment or prevention for AD when at least one SNP of step (a) is detected.
31. An assay for selecting a subject having or at risk for Alzheimer's disease, wherein subject is susceptible to treatment with a TS1 PLXNA4 inhibitory agent, the method comprising:
   a. subjecting a test sample from a subject to at least one genotyping assay that determines the genotypes of at least one (e.g., one, two, or three) loci, wherein said loci are selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof; and
   b. identifying Alzheimer's disease in the subject as susceptible for treatment with a TS1 PLXNA4 inhibitory agent when at least one of the following combinations of SNPs is determined to be present: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof.
32. An assay for selecting a subject having or at risk for Alzheimer's disease, wherein subject is susceptible to treatment with a TS1 PLXNA4 inhibitory agent, the method comprising:
   a. transforming at least one nucleic acid polymorphism in a locus in a biological sample from the subject into at least one detectable target, wherein the locus is selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof; and
   b. detecting presence or absence of at least one AD risk associated SNP from the at least one detectable target, wherein the at least one AD risk associated SNP is selected from: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof; and
   c. identifying Alzheimer's disease in the subject as susceptible for treatment with a TS1 PLXNA4 inhibitory agent when at least one AD risk associated SNP is detected in step (b).
33. An assay for selecting a subject having or at risk for Alzheimer's disease, wherein subject is susceptible to treatment with a TS1 PLXNA4 inhibitory agent, the method comprising:
   a. contacting a biological sample obtained from a subject with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. washing the sample to remove unbound oligonucleotide;
   c. measuring the intensity of the signal from the bound, detectable bound detectable oligonucleotide;
   d. comparing the measured intensity of the signal with a reference value and if the measured intensity is increased relative to the reference value; and
   e. identifying Alzheimer's disease in the subject as susceptible for treatment with a TS1 PLXNA4 inhibitory agent when at least one SNP of step (a) is detected.
34. An assay comprising:
   a. measuring or quantifying expression level or amount of TS1 or TS3 PLXNA4 in a biological sample obtained from a subject; and
   b. comparing the measured or quantified expression level or amount of TS1 or TS3 PLXNA4 with a reference value, and if the amount of expression level or amount of TS1 or TS3 PLXNA4 is increased relative to the reference value,
   c. identifying the subject as having an increased probability of having AD.
35. An assay comprising:
   a. transforming the gene expression product of TS1 or TS3 PLXNA4 transcript into a detectable target;
   b. measuring the amount of the detectable target;
   c. comparing the amount of the detectable target to a reference; and if the amount of the detectable target is higher than a reference level; and
   d. identifying the subject as having an increased probability of having AD
36. An assay comprising:
   a. contacting a biological sample obtained from a subject with a detectable antibody specific for TS1 or TS3 PLXNA4 or detectable nucleic acid for TS1 or TS3 PLXNA4;
   b. washing the sample to remove unbound antibody or unbound nucleic acid;
   c. measuring the intensity of the signal from the bound, detectable antibody or bound detectable nucleic acid;
   d. comparing the measured intensity of the signal with a reference value and if the measured intensity is increased relative to the reference value; and
   e. identifying the subject as having an increased probability of having AD.
37. An assay for identifying a subject having or at risk for Alzheimer's disease comprising:
   a. measuring or quantifying the level of expression or amount of TS1 or TS3 TSPLXNA4 transcript in a biological sample obtained from a subject; and
   b. identifying the subject as having or at risk for Alzheimer's disease if the amount of TS1 and/or TS3 PLXNA4 transcript is increased relative to a reference value.
38. An assay for determining an increased risk of a subject for developing Alzheimer's disease comprising
   a. transforming the gene expression product of TS1 or TS3 PLXNA4 transcript into a detectable target;
   b. measuring the amount of the detectable target;
   c. comparing the amount of the detectable target to a reference; wherein if the amount of the detectable target is higher than a reference level, the subject is at increased risk for developing AD.
39. An assay for determining an increased risk of a subject for developing Alzheimer's disease comprising:
   a. contacting a biological sample obtained from a subject with a detectable antibody specific for TS1 or TS3 PLXNA4 or detectable nucleic acid for TS1 or TS3 PLXNA4;
   b. washing the sample to remove unbound antibody or unbound nucleic acid;
   c. measuring the intensity of the signal from the bound, detectable antibody or bound detectable nucleic acid;
   d. comparing the measured intensity of the signal with a reference value and if the measured intensity is increased relative to the reference value; and
   e. identifying the subject as having an increased probability of having AD.
40. An assay for selecting a subject having or at risk for Alzheimer's disease, wherein subject is susceptible to treatment with a TS1 PLXNA4 inhibitory agent, the method comprising:
   a. measuring or quantifying the level of expression or amount of TS1 or TS3 TSPLXNA4 transcript in a biological sample obtained from a subject; and
   b. identifying Alzheimer's disease in the subject as susceptible for treatment with a TS1 PLXNA4 inhibitory agent when the amount of TS1 and/or TS3 PLXNA4 transcript is increased relative to a reference value.
41. An assay for selecting a subject having or at risk for Alzheimer's disease, wherein subject is susceptible to treatment with a TS1 PLXNA4 inhibitory agent, the method comprising:
   a. transforming the gene expression product of TS1 or TS3 PLXNA4 transcript into a detectable target;
   b. measuring the amount of the detectable target;
   c. comparing the amount of the detectable target to a reference; and
   d. identifying Alzheimer's disease in the subject as susceptible for treatment with a TS1 PLXNA4 inhibitory agent when the amount of the detectable target is higher than a reference level.
42. An assay for selecting a subject having or at risk for Alzheimer's disease, wherein subject is susceptible to treatment with a TS1 PLXNA4 inhibitory agent, the method comprising:
   a. contacting a biological sample obtained from a subject with a detectable antibody specific for TS1 or TS3 PLXNA4 or detectable nucleic acid for TS1 or TS3 PLXNA4;
   b. washing the sample to remove unbound antibody or unbound nucleic acid;
   c. measuring the intensity of the signal from the bound, detectable antibody or bound detectable nucleic acid;
   d. comparing the measured intensity of the signal with a reference value; and
   e. identifying Alzheimer's disease in the subject as susceptible for treatment with a TS1 PLXNA4 inhibitory agent when measured intensity of the signal from the bound, detectable antibody or bound, detectable nucleic acid is higher than a reference level.
43. An assay for identifying a subject having or at risk for Alzheimer's disease comprising:

a. measuring or quantifying the level of expression or amount of TS1 or TS3 PLXNA4 transcript in a first biological sample obtained from a subject; and

b. measuring or quantifying the level of expression level or amount of TS1 or TS3 PLXNA4 transcript in a second biological sample obtained from a subject; and
c. identifying the subject as having or at risk for Alzheimer's disease if the amount of Ts1 or TS3 PLXNA4 transcript is increased in the second biological sample relative to the first biological sample by at least 20%.
44. An assay for identifying a subject having or at risk for Alzheimer's disease comprising:
   a. measuring or quantifying the amount of TS3 PLXNA4 transcript and TS1 PLXNA4 transcript in a biological sample obtained from a subject; and
   b. identifying the subject as having or at risk for Alzheimer's disease if the amount of TS3 PLXNA4 transcript, TS1 PLXNA4 transcript, or both are increased relative to a reference TS3 PLXNA4 transcript value.
45. The assay of any of paragraphs 21-44, further comprising selecting the subject for a treatment regimen, if the subject is identified as having or at risk for Alzheimer's disease.
46. The assay of any of paragraphs 21-45, further comprising selecting the subject for administering a TS1 PLXNA4 inhibitory agent, if the subject is identified as having or at risk for Alzheimer's disease.
47. The assay of any one of paragraphs 21-46, wherein the biological sample is a serum sample.
48. A computer implemented system for determining presence or absence of alleles associated with an increased risk of a subject for developing late onset Alzheimer's disease (AD), the system comprising:
   a. a determination module configured to identify and detect at least one single nucleotide polymorphism (SNP) in a biological sample of a subject, wherein the SNP is selected from: (i) SNP1, wherein SNP1 is identified by rs277472 on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235, wherein SEQ ID NO. 1 is a portion of genomic nucleic acid sequence of PLXNA4; (iii) SNP3, wherein SNP3 identified by rs11761937 on SEQ ID NO: 1, wherein the SEQ ID NO. 1 is a portion of genomic nucleic acid sequence of PLXNA4; and (iv) any combinations thereof;
   b. a storage module configured to store output data from the determination module;
   c. a computing module adapted to identify from the output data at least one of AD risk associated SNP is present in the output data stored on the storage module, wherein the AD risk associated SNP is selected from: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof; and
   d. a display module for displaying if any of the AD risk associated SNP was identified or not, and/or displaying the detected alleles.
49. A computer readable medium having computer readable instructions recorded thereon to define software modules for implementing a method on a computer, said computer readable storage medium comprising:
   a. instructions for comparing the data stored on a storage device with reference data to provide a comparison result, wherein the comparison identifies the presence or absence of at least one of the following conditions: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
   b. instructions for displaying a content based in part on the data output from said determination module, wherein the content comprises a signal indicative of the presence of at least one of the conditions, and optionally the absence of one or more of the conditions

### SOME SELECTED DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein, “SEQ ID NO: 1” refers to the genomic sequence of human PLXNA4 of Gene ID 91584, which can be found at bases 131808091 to 132333447 of the human chromosome 7 NCBI Reference Sequence: NC_000007.13.

As used herein, the “TS1 PLXNA4 transcript” or “TS1” refers to the full-length Plexin A4 (PLXNA4) transcript that contains 31 exons and encodes an isoform with 1,894 residues. As used herein, “TΩ PLXNA4 transcript” or “TS2” and “TS3 PLXNA4 transcript” or “TS3” refer to two alternatively spliced transcripts each of which contains three exons, thereby yielding shorter isoforms of 522 residues (TS2) and 492 residues (TS3), respectively.

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis.

As used herein, the term “nucleic acid” or “oligonucleotide” or “polynucleotide” refers to a polymer or an oligomer of nucleotide or nucleoside monomers consisting of nucleobases, sugars and intersugar linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

As used herein, “effective treatment” includes any statistically significant improvement in one or more indicia of the disease or disorder.

As used herein, the terms “prevent,” “preventing” and “prevention” refer to the avoidance or delay in manifestation of one or more symptoms or measurable markers of a disease or disorder, e.g., Alzheimer's disease. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

The oligonucleotide can be single-stranded or double-stranded. A single-stranded oligonucleotide can have double-stranded regions and a double-stranded oligonucleotide can have single-stranded regions. The oligonucleotide can have a hairpin structure or have a dumbbell structure. The oligonucleotide can be circular, e.g., wherein the 5′end of the oligonucleotide is linked to the 3′ end of the oligonucleotide. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe that can hybridize to the target sequence under stringent hybridization conditions. Thus, a nucleic acid also encompasses a probe that hybridizes under stringent hybridization conditions.

The oligonucleotides described herein can comprise any oligonucleotide modification described herein and below. In some embodiments, the oligonucleotide comprises at least one modification. In some embodiments, the modification is selected from the group consisting of a sugar modification, a non-phosphodiester inter-sugar (or inter-nucleoside) linkage, nucleobase modification, and ligand conjugation.

As used herein, an oligonucleotide can be of any length. In some embodiments, oligonucleotides can range from about 6 to 100 nucleotides in length. In various related embodiments, the oligonucleotide can range in length from about 10 to about 50 nucleotides, from about 10 to about 35 nucleotides, from about 15 to about 30 nucleotides, from about 20 to about 30 nucleotides in length. In some embodiments, oligonucleotide is from about 8 to about 39 nucleotides in length. In some embodiments, the oligonucleotide is 6 to 25 nucleotides in length (e.g., 6, 7, 8, 9, 10, 11, 12, 13, 14, 15, 16, 18, 19, 20, 21, 22, 23, or 24 nucleotides in length). In some embodiments the oligonucleotide is 25-30 nucleotides. In some embodiments, the single-stranded oligonucleotide is 15 to 29 nucleotides in length. In some other embodiments, the oligonucleotide is from about 18 to about 25 nucleotides in length. In some embodiments, the oligonucleotide is about 23 nucleotides in length. In some embodiments, a single-stranded oligonucleotide is 6, 7, 8, 9, 10, 11, 12, 13, 14, 15, 16, 17, 18, or 19 nucleotides in length.

The oligonucleotide can be completely DNA, completely RNA, or comprise both RNA and DNA nucleotides. It is to be understood that when the oligonucleotide is completely DNA, RNA, or a mix of both, the oligonucleotide can comprise one or more oligonucleotide modifications described herein.

In some embodiments of the various aspect described herein, the oligonucleotides can include one or more oligonucleotide or nucleic acid modifications. Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

The phosphate group in the intersugar linkage can be modified by replacing one of the oxygens with a different substituent. One result of this modification to RNA phosphate intersugar linkages can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. Some exemplary intersugar linkage modifications include phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH2), diinethylene-sulfoxide (—CH₂—SO—CH₂), dimethylene-sulfone (—CH2-SO₂—CH₂), 2′-O-alkyl, and 2′-deoxy-2′-fluoro phosphorothioate.

An oligonucleotide can include modification of all or some of the sugar groups of the nucleic acid. For example, the 2′ position (H, DNA; or OH, RNA) can be modified with a number of different “oxy” or “deoxy” substituents. While not being bound by theory, enhanced stability is expected since the 2′-hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible. Preferred sugar modifications are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration” refers to the placement of a substituent on the C3′ of ribose in the same configuration as the 3′-OH is in the xylose sugar.

Adenine, cytosine, guanine, thymine and uracil are the most common bases (or nucleobases) found in nucleic acids. These bases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the above modifications. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage. Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylaminocarbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any modified or nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

The terms “subject” and “individual” are used interchangeably herein, and refer to an animal, for example a human, to whom treatment, including prophylactic treatment, with a pharmaceutical composition according to the present invention, is provided. The term “subject” as used herein refers to human and non-human animals. The term “non-human animals” and “non-human mammals” are used interchangeably herein, and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

A “pharmaceutical composition” refers to a composition that usually contains an excipient, such as a pharmaceutically acceptable carrier that is conventional in the art and that is suitable for administration to cells or to a subject. In addition, compositions for topical (e.g., oral mucosa, respiratory mucosa) and/or oral administration can be in the form of solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The terms “significantly different from,”, “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

The terms “increased,” “increase” or “enhance” in connection with the amount of TS3 or TS1 PLXNA4 transcript in a biological sample obtained from a subject are all used herein to generally mean an increase by a statically significant amount. For the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference value or level, or at least about a 1.5-fold, at least about a 1.6-fold, at least about a 1.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level. In some embodiments, an increase is at least one standard deviation greater than, or at least two standard deviations, or more, greater than a median or mean reference level. Such median or mean reference levels can be obtained, for example, from five or more samples obtained from subjects not having Alzheimer's disease, or from five or more samples obtained from the same subject at different timepoints.

In embodiments of the various aspects disclosed herein, the reference level can be obtained or measured in a reference biological sample, such as a reference sample obtained from an age-matched normal control (e.g., an age-matched subject not having Alzheimer's disease), or a reference sample from the same subject at an earlier timepoint, for example, a “first biological sample.” A “reference value” is thus, in some embodiments, a predetermined reference level, such as an average or median amount or level of TS3 or TS1 PLXNA4 transcript obtained from, for example, biological samples from a population of healthy subjects that are in the chronological age group matched with the chronological age of the tested subject.

In embodiments of the various aspects disclosed herein, the reference can be a normal healthy subject with no genetic susceptibility for AD. For example, a normal healthy subject is not a carrier of any of the late onset AD risk associated alleles described herein or is not diagnosed with any forms of AD such as early-onset autosomal-dominant AD, or any neurodegenerative disorders. The reference can be also a control sample, a pooled sample of control individuals or a numeric value or range of values based on the same.

As used herein, the terms “biological sample” or “subject sample” or “sample” refer to a quantity of tissue or fluid, or a cell or population of cells obtained from a subject. In some embodiments, the biological sample is a blood sample, including, for example, a serum sample, or a plasma sample. Most often, the sample has been removed from a subject, but the term “biological sample” can also, in some embodiments, refer to cells or tissue or a quantity of tissue or fluid analyzed in vivo, i.e. without removal from the subject. A biological sample or tissue sample includes, but is not limited to, blood, plasma, serum, cerebrospinal fluid, lymph fluid, bone marrow, tumor biopsy, urine, stool, sputum, pleural fluid, nipple aspirates, lymph fluid, the external sections of the skin, lung tissue, adipose tissue, connective tissue, sub-epithelial tissue, epithelial tissue, liver tissue, kidney tissue, uterine tissue, respiratory tissues, breast tissue, gastrointestinal tissue, and genitourinary tract tissue, tears, saliva, milk, cells (including, but not limited to, blood cells), biopsies, scrapes (e.g., buccal scrapes), tumors, organs, and also samples of an in vitro cell culture constituent. Often, a “biological sample” can comprise cells from the subject, but the term can also refer to non-cellular biological material, such as non-cellular fractions of blood, saliva, or urine.

The term “reduced” or “reduce” or “decrease” as used herein generally means a decrease by a statistically significant amount relative to a reference. However, for avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e. absent level as compared to a reference sample), or any decrease between 10-100% as compared to a reference level, as that term is defined herein.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Further than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The disclosure is further illustrated by the following examples which should not be construed as limiting. The examples are illustrative only, and are not intended to limit, in any manner, any of the aspects described herein. The following examples do not in any way limit the invention.

### Examples

Alzheimer's disease is the most frequent age-related dementia affecting 5.4 million Americans including 13% of people ages 65 and older and over 40% of people ages 85 and older.¹ Genetic factors account for much of the risk for developing AD with heritability estimates between 60% and 80%.² The apolipoprotein E (APOE) ε4 allele is a well-recognized major risk factor for late onset AD, increasing the odds of disease in a dose-dependent fashion.³ Common polymorphisms in ten additional genes have been robustly established as risk factors for AD using large-scale genome-wide association studies (GWAS) and meta-analyses.^{4,5} These polymorphisms link to mechanisms of AO metabolism, lipid metabolism, inflammation, and axon guidance.^{6,7} However, the heritability of AD explained by APOE is about 17.5% and by each of the novel GWAS loci is less than 1%, suggesting that less than 30% of the genetic contribution to AD is explained by known common polymorphisms.^{4,8} The remaining heritability may be due to additional common variants of weaker effect, rare variants, copy-number variants, insertion-deletion polymorphisms, and gene-gene and gene-environment interactions.^{9,10}.

As described herein, we conducted a two-stage family-based AD GWAS using a novel method which incorporates the entire family structure and reduces diagnostic misclassification in the association test, and renders a result that is less prone to type I error, even for rare variants.¹¹ We obtained genome wide significant evidence of association with PLXNA4, a gene which has not been previously linked to AD. Subsequent in silico and molecular studies indicated that intronic polymorphisms affect alternative transcription of PLXNA4 isoforms. We observed isoform-specific effects of PLXNA4 on hyperphosphorylation of tau protein, a terminal step leading to breakdown of neuronal signaling and microtubule formation.

### Methods

Subjects: The GWAS was performed using a community-based sample consisting of 61 incident AD cases and 2,530 cognitively normal controls from 1,232 families in the Framingham Heart Study (FHS). The top findings were evaluated in a second cohort including 1,819 AD cases and 1,969 controls from 2,265 families containing multiple members affected by AD in the National Institute on Aging—Late Onset Alzheimer Disease (NIA-LOAD) Study. Clinical, demographic, pedigree and genetic data for both cohorts were obtained from dbGaP on the worldwide web at www.ncbi.nlm.nih.gov/gap. Additional details about the ascertainment, evaluation and characteristics of these subjects are provided.

Statistical Analysis:

Details of the statistical analysis, including a comprehensive description of procedures for quality control of the GWAS data, evaluation of population substructure, genotype imputation, and genetic association and bioinformatics methods are available.

Amyloid Precursor Protein (APP), Amyloid β (Aβ) and Tau Analyses:

Methods for investigating the effect of PLXNA4 on the processing of APP, Aβ and tau protein are presented.

Analyses of Gene Expression in Brain:

Gene expression experiments were conducted on brain tissue specimens obtained from 17 autopsied subjects including five controls (Braak stage 0), five early-stage AD cases (Braak stages 1-2), and nine late-stage AD cases (Braak stages 3-4) in the Mount Sinai/Bronx Veterans Administration (VA) Medical Center/Department of Psychiatry Brain Bank. Normal controls had no history of any psychiatric or neurological disorders and no discernible neuropathological lesions. Ascertainment, cognitive assessment, neuropathological assessment, and stratification of these subjects were previously described.¹² The Institutional Review Boards of Pilgrim Psychiatric Center, Mount Sinai School of Medicine, and the Bronx Va. Medical Center approved all assessment and post-mortem procedures. Additional subject details and procedures for quantification of PLXNA4 expression are provided.

Analyses of Gene Expression in Blood:

The correlation of PLXNA4 SNP genotypes and expression in blood was evaluated in a sample of 116 cognitively healthy Korean volunteers ages 11-45 (mean=28.2+6.0 years).¹³ Details of DNA and RNA preparation, PLXNA4 isoform quantification, SNP genotyping and statistical analysis of these data are available.

### Results and Discussion

The mean onset age of AD among the 61 incident cases in the FHS dataset was about 10 years older than that for the 1,819 AD cases in the NIA-LOAD database. The frequency of the APOE ε4 allele in unaffected subjects (FHS: n=2,530, NIA-LOAD: n=1,969) was comparable between the two family datasets, but the ε4/ε4 genotype frequency was approximately nine times greater in AD subjects in the NIA-LOAD cohort compared to AD subjects in the FHS cohort. In addition, the proportion of AD subjects lacking ε4 was about three times larger in the FHS cohort. Among the unaffected family members in the NIA-LOAD cohort, 439 had propensity scores of at least 80% (i.e., risk to develop AD accounting for parental affection status and age at onset/exam and adjusting for population substructure) and 809 had propensity scores of zero.

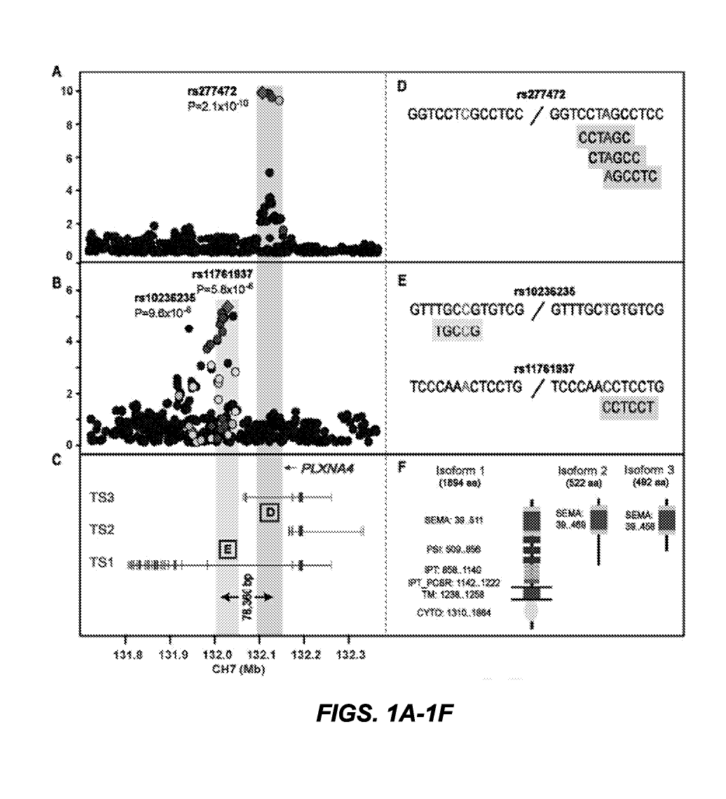

Analysis of the 341,492 genotyped SNPs in the FHS discovery sample revealed strong evidence of association with AD in multiple regions of the genome (Table 1). The distribution of observed p-values for the entire set of SNPs indicated little genomic inflation (X=1.01). Genome-wide significant associations were found with SNPs in ITIH3 (rs9311482: (β=1.3, P=5×10⁻⁹), PLXNA4 (rs277484: β=1.1)P=9×10⁻¹° and MYO18B (rs13057714: β=1.0, P=9×10⁻⁹) (Table 2). SNPs located in IGSF21 and between GPR149 and MME were also significant at P<10⁻⁶. Each dose of the minor alleles for these SNPs increased AD risk by at least one unit (mean liability on the normalized scale). We attempted to discover additional association signals in IGSF21, ITIH3, PLNXA4, and MYO18B by evaluating 1,515 genotyped and accurately imputed SNPs (RSQ≧0.8) in the FHS datasets. One other IGSF21 SNP and five additional PLNXA4SNPs were significant after adjustment for multiple testing (P<3.3×10⁻⁵). The most notable result was obtained for PLNXA4 SNP rs277472 (P=2.0×10⁻¹⁰) (FIG. 1A). No additional SNPs from the ITIH3 or the MYO18B met the significance threshold after multiple testing corrections.

Next, we tested association of 1,502 genotyped and accurately imputed SNPs (RSQ≧≧8) SNPs in IGSF2, ITIH3, PLXN, 44, and MYO18B in the NIA-LOAD dataset (Table 1). Further analysis revealed significant association of the AD propensity score with ten other PLXNA4 SNPs after multiple testing correction (SNPs with P<10⁻⁵: rs11761937, (3=−0.09, P=5.8×10⁻⁶ and rs10236235, (3=−0.07, P=9.7×10⁻⁶) in the NIA-LOAD dataset (FIG. 1B). Genotyped SNP rs11761937 and imputed SNP rs10236235 are in complete linkage disequilibrium (LD, r²=1.0). Each dose of the minor allele T for rs11761937 accounts for a 9% reduction in disease penetrance. These results were not meaningfully changed after adjustment for sex and the number of APOE ε4 alleles, although there was evidence for interaction between APOE and two SNPs on AD susceptibility (rs11761937: (3INT=0.07, PINT=0.012; rs10236235: (3INT=0.07, PINT=0.0028),

Of 337 SNPs from IGSF21, PLXNA4, and MYO18B common to both datasets, five PLXNA4 SNPs were significant in the combined datasets after multiple testing correction, most notably rs10236235 (FHS: (3=−0.13, P=0.1 1; NIA-LOAD: (3=−0.07, P=9.7×10⁻⁶; total: (3=−0.07, P=4.4×10⁻⁷). This result was not meaningfully different (combined P=4.2×10⁻⁶) in the meta-analysis using the total number of affected subjects in each study. Gene-based analyses including the set of 337 SNPs confirmed association with PLXNA4 in the FHS (p=0.004), NIA-LOAD (p=0.03), and combined (p=3.6×10⁻⁴) datasets (Table 2).

PLXNA4 is a member of a family of receptors for transmembrane, secreted and GPI-anchored semaphorins in vertebrates¹⁴ and is a receptor for secreted semaphorin class 3 (SEMA3A) and class 6 (SEMA6) proteins which play an important role in semaphorin signaling and axon guidance.¹⁵ Accumulation of SEMA3A was previously detected in susceptible areas of the hippocampal neurons during AD progression and colocalized with phosphorylated tau.¹⁶ A phosphorylated CRMP2 protein, an intracellular signaling molecule for the semaphorin-plexin signaling pathway, has been observed in neurofibrillary tangles in brains of autopsied AD patients.¹⁷ The staining pattern of SEMA6, which is present in fibers and nerve terminals, is disrupted in brains of patients with AD.¹⁸

There are three known alternatively spliced PLXNA4 transcripts. As used herein, the “TS1 PLXNA4 transcript” or “TS1” is the full-length PLXNA4 transcript that contains 31 exons and encodes an isoform with 1,894 residues. As used herein, “TS2 PLXNA4 transcript” or “TS2” and “TS3 PLXNA4 transcript” or “TS3” refer to two alternatively spliced transcripts each of which contains three exons yielding shorter isoforms of 522 residues (TS2) and 492 residues (TS3), respectively. As shown herein, the distinct PLXNA4 association peaks in the discovery and replication datasets flank an exon that is present only in TS3.

Further scrutiny of the PLXNA4 findings revealed that the most significant SNPs in each dataset clustered in distinct regions approximately 78,360 base pairs apart in intron 2 of the largest transcript (TS1) and flank an alternatively spliced exon present only in a much shorter transcript (TS3) (FIG. 1C). We screened the DNA sequences surrounding the SNPs most significantly associated with AD susceptibility in each dataset for intronic splicing regulatory elements (IRE) that might be impacted by an allelic difference at the site of the SNP. The sequence surrounding the most significantly associated SNP in the FHS dataset (rs277472) contained three IRE motifs in the presence of the risk allele A, but none if the alternative allele was there instead (FIG. 1D). In contrast, one IRE motif was identified in the sequence surrounding the most significantly associated SNP in the combined dataset (rs10236235) but only in the presence of the protective allele T (FIG. 1E). Other AD-associated SNPs were also predicted to impact splicing. Bioinformatic evaluation confirmed that the longer isoform contains a transmembrane domain, while the shorter forms are predicted as secreted forms (FIG. 1F), indicating that different isoforms can have distinct functional consequences related to AD.

To determine whether PLXNA4 isoforms are differentially involved in APP processing and Aβ production, we transfected one of the three isoforms PLXNA4-Myc into HEK293 cells stably expressing APP751 and analyzed total APP in cell lysates and APPsα and Aβ 40 and 42 in the medium. Neither over-expressing PLXNA4 isoforms affected APPsα secretion or Aβ 40 and 42 production. These results indicate that PLXNA4 is not involved in AD through the α- or β-secretase pathways.

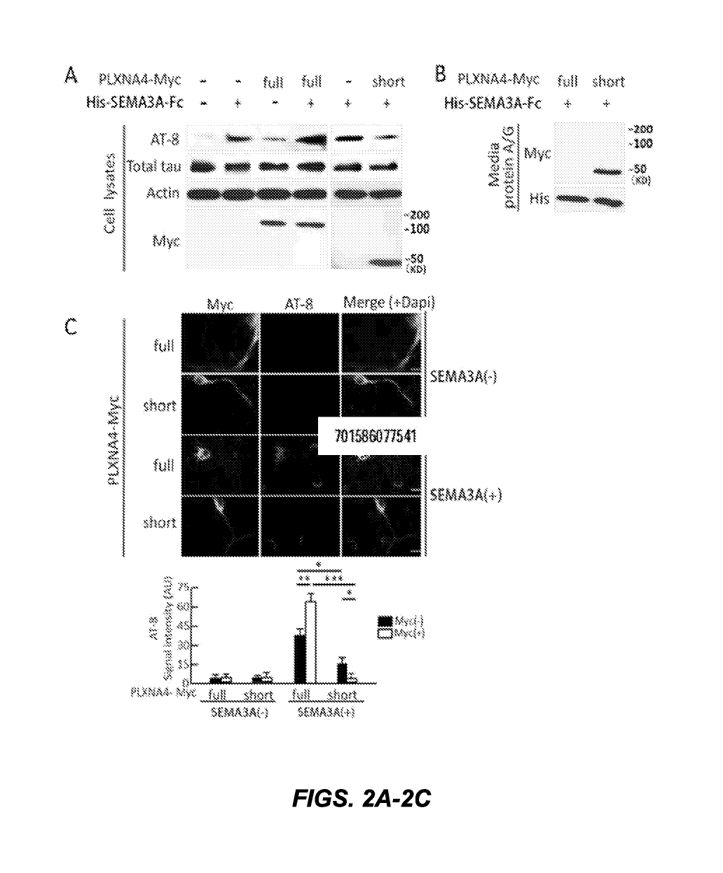

Involvement of plexin-A4 signaling in tau phosphorylation was examined by transfecting cDNAs for the full-length (TS1) or 3′ C-terminal truncated short isoforms (TS2 and TS3) of human PLXNA4-Myc into SH-SY5Y P301L cells (SH-SY5Y cells stably expressing the P301L tau mutant) and stimulated with or without 3 nM recombinant Semaphorin-3A (SEMA3A-FC). Immunoblotting with AT8 (anti-phospho-Tau at Ser²⁰²/Thr²⁰⁵) showed that tau phosphorylation was induced by SEMA3A stimulation, which was enhanced by over-expression of TS1 (FIG. 2A). In contrast, over-expression of either TS2 or TS3 inhibited tau phosphorylation under stimulation by SEMA3A. Since TS2 and TS3 are secretory molecules with the SEMA3A binding site in the extracellular domain, we assume that the inhibitory effect is mediated by the competitive binding of the short isoforms to SEMA3A. Pull-down assays confirmed that the short isoforms, but not full-length PLXNA4, specifically co-precipitated with SEMA3A in the media (FIG. 2B). These data demonstrate that the short isoforms are secreted as expected and bind to SEMA3A thereby inhibiting signaling. Further examination in rat primary hippocampal neurons illustrated that transient expression of Myc-tagged full-length PLXNA4 elevated SEMA3A-induced tau phosphorylation (Myc⁺ AT8⁺ cells, red staining, FIG. 2C), while expression of Myc-tagged shorter isoforms reduced SEMA3A-induced tau phosphorylation in neurons.

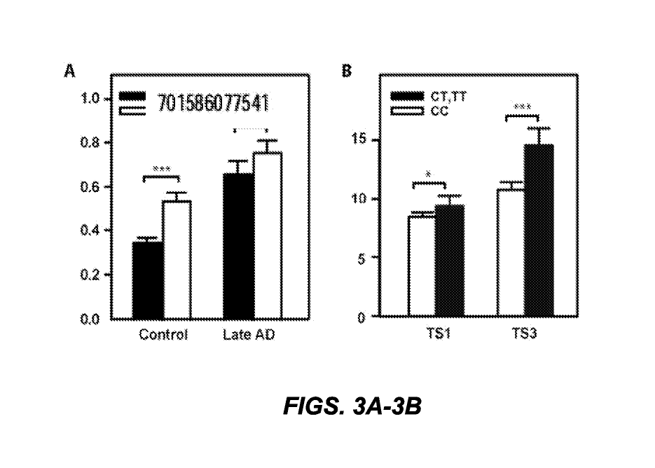

Expression of the TS1 and TS3 was quantified in brain tissue specimens from the middle frontal gyms (Brodmann area 9) from 19 autopsied subjects including five controls, five early-stage AD cases, and nine late-stage AD cases. Late-stage AD cases compared to controls had 1.9-fold increased expression of TS1 (P=6.0×10⁻⁴) and a more modestly increased expression of TS3 (P=0.021) (Table 3, FIG. 3A). These patterns were similar in the comparison of early-stage AD cases to controls (Table 3) and are not age-related. In the combined sample of AD cases and controls, TS1 level was significantly correlated with the clinical dementia rating score (r²=0.75, P=2.2×10⁻⁴) and several measures of AD neuropathology (r²˜0.5, P<0.05), but the correlations of TS3 level with these traits were much smaller. These findings indicate that elevation in TS1 level increases risk for developing AD.

Association of five significantly associated PLXNA4 SNPs with expression of the TS1 and TS3 isoforms was evaluated in serum from 116 young healthy controls. Expression levels were significantly lower for TS3 (p=8.62×10⁻⁹) and TS1 (p=0.024) among individuals homozygous for the protective rs1593222 C allele under a dominant model (FIG. 3B). A similar but less significant trend was noted for the additive model. Similar patterns were observed with other SNPs, most notably with rs6959579 (p=1.89×10⁻⁵) and rs17166338 (p=0.0095) for TS3 under a dominant model, but the strength of the association may have been lower with some SNPs because of fewer homozygotes for the protective alleles. These results indicate that possession of at least one copy of the alternate allele, which is the AD risk allele for each of these SNPs, is correlated with higher levels of TS3 and possibly TS1 in serum decades before AD typically occurs.

As demonstrated herein, we identified significant association between AD susceptibility and SNPs in PLXNA4 using a family-based approach. The top-ranked SNPs in the discovery and replication datasets are located in a single intron and surround an exon that is skipped in the processing of the full-length mRNA transcript. We also demonstrated that the full-length isoform (TS1), but not the shorter isoforms (TS2 and TS3), of PLXNA4 (the protein encoded by PLXNA4) increased tau phosphorylation in SH-SY5Y cells stably expressing the P301L tau mutant and in primary rat neurons when stimulated by SEMA3A. Significantly higher levels of TS1 and TS3 in cortical tissue were observed in late-stage AD cases compared to controls. By comparison, transfection of either isoform into HEK293 cells stably expressing APP failed to show differential effects on APP processing or Aβ production. Taken together, our results indicate that PLXNA4-mediated tau phosphorylation is an independent upstream event leading to AD-related tangle formation in neurons.

PLXNA4 is a member of a family of receptors for transmembrane, secreted and GPI-anchored semaphorins in vertebrates¹⁴ and is a receptor for secreted semaphorin class 3 (SEMA3A) and class 6 (SEMA6) proteins which play an important role in semaphorin signaling and axon guidance.¹⁵ Accumulation of SEMA3A was previously detected in susceptible areas of the hippocampal neurons during AD progression and colocalized with phosphorylated tau.¹⁶ A phosphorylated CRMP2 protein, an intracellular signaling molecule for the semaphorin-plexin signaling pathway, has been observed in neurofibrillary tangles in brains of autopsied AD patients.¹⁷ The staining pattern of SEMA6, which is present in fibers and nerve terminals, is disrupted in brains of patients with AD.¹⁸ These reports and our study collectively indicate that disrupted semaphorin-plexin signaling is involved in AD pathogenesis, specifically through tau phosphorylation leading to tangle formation and neuronal death.

There are three known alternatively spliced PLXNA4 transcripts. The full-length transcript (TS1) contains 31 exons and encodes an isoform with 1,894 residues. Two alternatively spliced transcripts each contain three exons yielding shorter isoforms of 522 residues (TS2) and 492 residues (TS3), respectively. The distinct PLXNA4 association peaks in the discovery and replication datasets flank an exon that is present only in TS3. Our bioinformatic analysis identified predicted intronic splicing regulatory elements near the most strongly associated SNP in each dataset. However, our finding of increased expression of both TS1 and TS3 indicate that the genetic mechanism can also involve transcription regulatory elements.

Our experiments using a neuronal cell line and rat primary neurons suggest that the longer transmembrane isoform of PLXNA4 increases tau phosphorylation while shorter secreted isoforms inhibit the effect. The specific findings supporting this conclusion include SEMA3A induced phosphorylation of tau, expression of full-length PLXNA4 coupled to SEMA3A enhanced tau phosphorylation, and the shorter isoforms binding to SEMA3A and blocking SEMA3A/PLXNA4 signaling for tau phosphorylation. Semaphorin-plexin signaling is known to regulate axon guidance in the development of sympathetic nervous system and cerebral cortex.¹⁹⁻21 Previously, binding of SEMA3A to truncated PLXNA proteins was demonstrated to have a dominant negative effect on cortical growth cone collapse.²² Our data demonstrate that disruption of this signaling can also contribute to the acceleration of tau phosphorylation leading to neurofibrillary tangle formation. Taken together, our findings point to a novel mechanism for AD-related tangle formation, implying that reduced expression of PLXNA4, and the TS1 isoform in particular, in brain is crucial to maintain healthy neurons.

A direct link of PLXNA4 expression to AD is supported by the evidence described herein of increased expression of TS1 and TS3 isoforms in post-mortem neuronal tissue from AD cases compared to controls. Importantly, the findings that the relative increase of TS1 is much greater than TS3 in AD cases, and TS1 expression is significantly correlated with clinical and neuropathological severity measures of AD, are consistent with our observation of increased phosphorylation of tau by SEMA3A bound specifically to TS1. In addition, our findings in sera from a group of young controls suggest that differential expression of these isoforms may be genetically regulated beginning in early life.

Previous GWAS and candidate gene studies involving the FHS and NIA-LOAD datasets have successfully identified several AD genes including SORL1, BIN1, MS4A4/MS4A6A, EPHA7, ABCA7, CD33, and CD2AP),^{4,5,23-25} but the portion of evidence for these associations attributable to either of these datasets is relatively small and the robust associations in these loci are with common SNPs (MAF>0.1). We increased the potential for gene discovery in the FHS and NIA-LOAD datasets by applying an analytical method that leverages the family structure that is otherwise ignored when using generalized estimating equations or sibpair-based analysis. This method is applicable to extended pedigrees and is robust for detecting association with less common SNPs (0.01≦MAF<0.1).¹¹ In addition, we applied a novel approach to address the differences between the FHS and NIA-LOAD datasets. We substituted a quantitative measure (propensity score) of disease liability for AD status in the NIA-LOAD dataset, which has a skewed distribution of age at onset and is enriched for familial AD in comparison with the community-based FHS sample. Our method using propensity scores as a surrogate for AD susceptibility assigned a >50% probability for future development of AD to 60% of the cognitively normal subjects in the NIA-LOAD cohort. This indicates that analyses using affection status in families with multiple affected members have much less power to detect true associations without adjusting for misclassification of unaffected relatives.

Our gene-based test results indicate that the significant results with individual PLXNA4 SNPs are not false positive findings. Since subjects in both studies are Caucasians of European origin, the MAFs for the top-ranked SNPs are similar across datasets and there is no LD (r²<0.2) among SNPs from the two association peaks, a more likely explanation for the different association patterns is allelic heterogeneity. Another concern is that the in vitro tau phosphorylation experiments do not indicate which tau kinases activate tau phosphorylation. Cdk5 and glycogen synthase kinase-3β are known to be activated by SEMA3A by phosphorylating CRMP2 to mediate growth cone collapse.^{26,27} Since these are established tau kinases, it is most likely that PLXNA4 induces tau phosphorylation via activation of these two kinases. Also, because these results are based on the transient expression of PLXNA4 molecules, it is crucial to evaluate the influence of PLXNA4 on tau phosphorylation in more physiological conditions. Isoform-specific gene-targeting of PLXNA4 in mice is a potential future study in vivo.

In summary, our novel genetic association findings and results of molecular and cell biology experiments in cell lines, rat neurons, and human blood and brain demonstrate that PLXNA4 is involved in AD pathogenesis. Evidence supporting transcriptional regulation of PLXNA4 isoforms that have differential effect on tau phosphorylation and, hence, tangle formation indicates a new drug target.

Replication and Extended Analysis of PLXNA4:

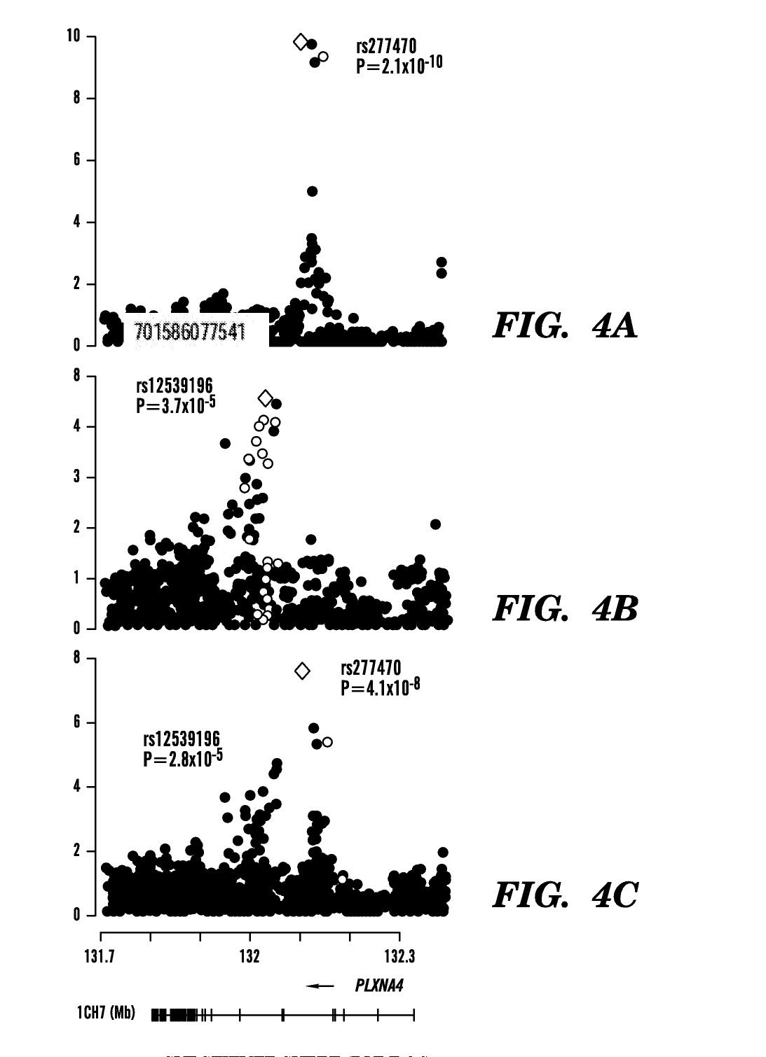

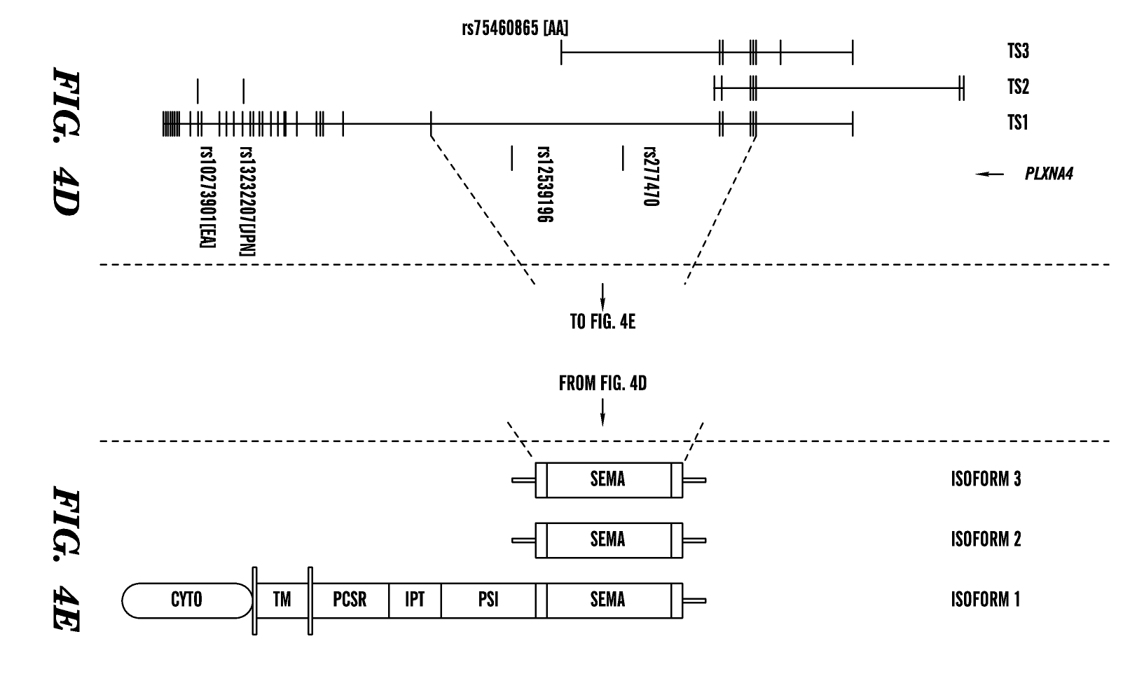

We tested association of 746 genotyped and accurately imputed SNPs (RSQ≧0.8) from PLXNA4 in the NIA-LOAD dataset (FIG. 4B), but were unable to replicate any of the top-ranked SNPs obtained in the FHS sample (Table 3). However, 16 of the 746 PLXNA4 SNPs showed significant association at P<10⁻³ in a model using the normalized liability score, and these association signals were improved using the normalized propensity score (Table 3). We observed an association trend in the same effect direction with rs277470 in the NIA-LOAD using the propensity score (p value: FHS=2.1×10⁻¹°, NIA-LOAD=0.06, meta-analysis=4.1×10⁻⁸) (FIG. 4C). Each dose of the minor allele C for rs277470 increased the liability rank by at least one unit in FHS dataset and the propensity rank by 13% in NIA-LOAD (Table 2). The most significant finding in NIA-LOAD (FIG. 4B) was obtained with a genotyped SNP rs12539196 (p value: FHS=0.114, NIA-LOAD=3.7×10⁻⁵, meta-analysis=2.8×10⁻⁵). The minor allele C for rs12539196 decreased the liability rank by 0.09 in FHS and accounts for a 15% reduction in the propensity rank in NIA-LOAD (Table 2). The result for rs12539196 in the NIA-LOAD dataset was not meaningfully changed after adjustment for the number of APOE ε4 alleles (P=2.7×10⁻⁵) and remained significant after correction for multiple testing (P=0.0036). Further scrutiny of the PLXNA4 findings revealed that the most significant SNPs in each dataset are clustered in two distinct regions approximately 78,240 base pairs apart in intron 2 of the largest transcript (TS1) and flank an alternatively spliced exon present only in a much shorter transcript (TS3) (FIG. 4D). Bioinformatic evaluation revealed that TS1 contains a transmembrane domain, whereas the shorter isoforms are predicted to be secreted (FIG. 4E), suggesting that the longer and shorter isoforms may have distinct functional consequences related to AD. Based on this information, we performed region-based analyses including only SNPs located between 131,925,825 and 132,193,452 base pairs (including all of intron 2) encompassing the SEMA domain (FIG. 4E), and confirmed significant association for the region (Table 2) in both datasets (P value: FHS=6.3×10⁻³; NIA-LOAD=0.019; meta-analysis=3.2×10⁻⁴).

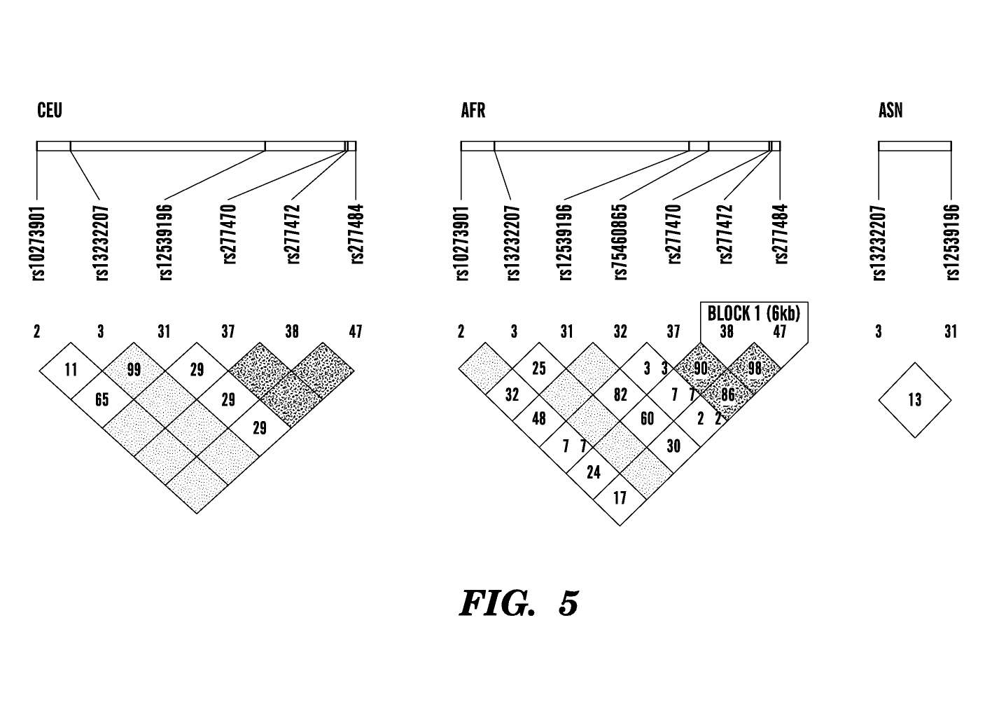

Association with PLXNA4 was further examined using summarized results from previous GWA studies reported by the Alzheimer's Disease Genetics Consortium (ADGC) for 18,901 Caucasians (9,966 cases and 8,935 controls) excluding the NIA-LOAD dataset,^{4,7} 4,896 African Americans (1,459 cases and 3,437 controls) excluding the NIA-LOAD dataset,²⁷ and 1,845 Japanese (951 cases and 894 controls).²⁸ The most significant SNPs in each population under the additive model were rs10273901 in Caucasians (minor allele frequency [MAF]=0.42; meta-analysis p value [meta-P]=3.9×10⁻⁵; odds ratio [OR]=0.85, 95% Confidence Interval [95% CI]: 0.79-0.92), rs75460865 in African Americans (MAF=0.04; meta-P=8.0×10⁻⁴; OR=1.55, 95% CI: 1.20-2.01), and rs13232207 in Japanese (MAF=0.19; P=1.2×10⁻⁴; OR=1.51, 95% CI: 1.22-1.86). The results for the Caucasian and Japanese groups remained significant after correcting for multiple testing (P=0.013 and P=0.037, respectively). Rs75460865 is located in the portion of the sequence which encodes the SEMA domain, but rs10273901 and rs13232207 are located in the PLXNA4 region that encodes the CYTO domain (FIG. 4E). Top ranked SNPs in the FHS and NIA-LOAD datasets were not associated in the other ADGC datasets (P>0.1) likely because power to detect association with several SNPs (including rs277470) having low minor allele frequencies is weaker in the ADGC datasets compared to large extended family-based samples. Because the LD structure in this region is similar across populations (FIG. 5), different association peaks among these groups is consistent with the existence of multiple distinct functionally-relevant AD-related alleles. In summary, the results in each of the ADGC ethnic samples support the association of AD with PLXNA4 SNPs.

### REFERENCES

1. Thies W, Bleiler L. 2011 Alzheimer's disease facts and figures Alzheimers Dement 2011; 7:208-244
2. Farrer L A. Genetics and the dementia patient. Neurologist 1997; 3:13-30
3. Farrer L A, Cupples L A, Haines J L, et al. Effects of age, sex, and ethnicity on the association between apolipoprotein E genotype and Alzheimer disease. A meta-analysis. APOE and Alzheimer Disease Meta Analysis Consortium. JAMA 1997; 278:1349-1356
4. Naj A C, Jun G, Beecham G W, Wang L S, et al. Common variants at MS4A4/MS4A6E, CD2AP, CD33 and EPHAl are associated with late-onset Alzheimer's disease. Nat Genet 2011; 43:436-441
5. Hollingworth P, Harold D, Sims R, et al. Common variants at ABCA7, MS4A6A/MS4A4E, EPHAl, CD33 and CD2AP are associated with Alzheimer's disease. Nat Genet 2011; 43:429435
6. Lill C M, Bertram L. Towards unveiling the genetics of neurodegenerative diseases. Semin Neurol 2012; 31:531-541
7. Paulson H L, Igo I. Genetics of dementia. Semin Neurol 2012; 31:449-460
8. So H C, Gui A H, Cherry S S, Sham P C. Evaluating the heritability explained by known susceptibility variants: a survey of ten complex diseases. Genet Epidemiol 2011; 35:310-317
9. Manolio T A, Collins F S, Cox N J, et al. Finding the missing heritability of complex diseases. Nature 2009; 461:747-753
10. Sherva R, Farrer L A. Power and pitfalls of the genome-wide association study approach to identify genes for Alzheimer's disease. Curr Psychiatry Rep 2011; 13:138-146
11. Choi S H, Liu C, Dupuis J, Logue M W, Jun G. Using linkage analysis of large pedigrees to guide association analyses. BMC Proc 2011; 5 Suppl 9:S79
12. Haroutunian V, Katsel P, Schmeidler J. Transcriptional vulnerability of brain regions in Alzheimer's disease and dementia. Neurobiol Aging 2009; 30:561-573.
13. Kim D H, Lee S-T, Won H-H, et al. A genome-wide association study identifies novel loci associated with susceptibility to chronic myeloid leukemia. Blood 2011; 117:6906-6911
14. Tamagnone L, Artigiani S, Chen H, et al. Plexins are a large family of receptors for transmembrane, secreted, and GPI-anchored semaphorins in vertebrates. Cell 1999; 99:71-80
15. Suto F, Ito K, Uemura M, et al. Plexin-a4 mediates axon-repulsive activities of both secreted and transmembrane semaphorins and plays roles in nerve fiber guidance. J Neurosci 2005; 25:3628-3637
16. Good P F, Alapat D, Hsu A, et al. A role for semaphorin 3A signaling in the degeneration of hippocampal neurons during Alzheimer's disease. J Neurochem 2004; 91:716-736
17. Cole A R, Knebel A, Morrice N A, et al. GSK-3 phosphorylation of the Alzheimer epitope within collapsin response mediator proteins regulates axon elongation in primary neurons. J Biol Chem 2004; 279:50176-50180
18. Hirsch E, Hu L J, Prigent A, et al. Distribution of semaphorin IV in adult human brain. Brain Res 1999; 823:67-79
19. Waimey K E, Huang P H, Chen M, Cheng H J. Plexin-A3 and plexin-A4 restrict the migration of sympathetic neurons but not their neural crest precursors. Dev Biol 2008; 315:448-458.
20. Sasaki Y, Cheng C, Uchida Y, et al. Fyn and Cdk5 mediate semaphorin-3A signaling, which is involved in regulation of dendrite orientation in cerebral cortex. Neuron 2002; 35:907-920.
21. Low L K, Liu X B, Faulkner R L, Coble J, Cheng H J. Plexin signaling selectively regulates the stereotyped pruning of corticospinal axons from visual cortex. Proc Natl Acad Sci USA 2008; 105:8136-8141.
22. Takahashi T, Fournier A, Nakamura F, et al. Plexin-Neuropilin-1 complexes form functional Semaphorin-3A receptors. Cell 1999; 99:59-69.
23. Harold D, Abraham R, Hollingworth P, et al. Genome-wide association study identifies variants at CLU and PICALM associated with Alzheimer's disease. Nat Genet 2009; 41:10881093
24. Rogaeva E, Meng Y, Lee J H, et al. The neuronal sortilin-related receptor SORL1 is genetically associated with Alzheimer disease. Nat Genet 2007; 39:168-177
25. Seshadri S, Fitzpatrick A L, Ikram M A, et al. Genome-wide analysis of genetic loci associated with Alzheimer disease. JAMA 2010; 303:1832-1840
26. Brown M, Jacobs T, Eickholt B, et al. Alpha2-chimaerin, cyclin-dependent Kinase 5/p 35, and its target collapsin response mediator protein-2 are essential components in semaphorin 3A-induced growth-cone collapse. J Neurosci 2004; 24:8994-9004.
27. Uchida Y, Ohshima T, Sasaki Y, et al. Semaphorin3A signalling is mediated via sequential Cdk5 and GSK3beta phosphorylation of CRMP2: implication of common phosphorylating mechanism underlying axon guidance and Alzheimer's disease. Genes Cells 2005; 10:165179.

TABLE 1
Association Results for AD Using Genoytped SNPs in the FHS and NIA-Load datasets

| | | | | | FHS | | | NIA-LOAD | | | Meta-Analysis | |
|---|---|---|---|---|---|---|---|---|---|---|---|---|
| SNP | CH | POS | GENE | MA | MAF | β | P | MAF | β | P | β | P |
| rs4920448 | 1 | 18,504,791 | IGSF21 | C | 0.032 | 0.52 | 2.3 × 10⁻⁷ | 0.044 | 0.09 | 0.373 | 0.30 | 2.2 × 10⁻⁵ |
| rs9311482 | 3 | 52,838,932 | ITIH3 | A | 0.010 | 1.34 | 4.6 × 10⁻⁹ | 0.028 | −0.01 | 0.943 | 0.49 | 4.8 × 10⁻⁴ |
| n16824295 | 3 | 154,509,114 | GPR149/MME | C | 0.011 | −1.00 | 4.1 × 10⁻⁷ | 0.025 | 0.26 | 0.047 | −0.13 | 0.252 |
| rs277484 | 7 | 132,119,654 | PLXNA4 | G | 0.011 | 1.06 | 9.0 × 10⁻¹⁰ | 0.012 | 0.15 | 0.289 | 0.51 | 2.8 × ¹⁰⁻⁶ |
| n13057714 | 22 | 26,362,534 | MYO18B | T | 0.013 | 1.02 | 8.9 × 10⁻⁹ | 0.011 | 0.00 | 0.993 | 0.36 | 5.9 × 10⁻⁴ |

CH: chromosome;
POS: base pair position from build 37;
MA: minor allele;
MAF: minor allele frequency;
β: estimate from the regression model;
P: P value in each study or in meta-analysis

TABLE 2
SNP-based and gene-based association results with genotyped and imputed SNPs
for AD risk in the FHS dataset and for AD propensity score in the NIA-LOAD dataset.

| | | | FHS | | | LOAD | | | Meta-Analysis | |
|---|---|---|---|---|---|---|---|---|---|---|
| Gene or SNP | CH:POS | RA or nSNPs | RAF | β | P | RAF | β | P | B or Zscore | P |
| IGF21 | 1: 18,446,816 | C | 0.98 | 0.156 | 0.341 | 0.98 | 0.033 | 0.031 | 0.034 | 0.015 |
| rs1275684 | | 46 | NA | NA | 0.563 | NA | NA | 0.251 | 1.22 | 0.222 |
| Gene-based | | | | | | | | | | |
| IGF21 | 7: | C | 0.94 | 0.131 | 0.116 | 0.94 | 0.068 | 3.6 × 10⁻⁴ | 0.070 | 4.4 × 10⁻⁷ |
| rs1275684 | 132,006,366 | 162 | NA | NA | 0.004 | NA | NA | 0.031 | 3.57 | 3.6 × 10⁻⁴ |
| Gene-based | | | | | | | | | | |
| IGF21 | 22: | C | 0.02 | | 0.121 | 0.02 | 0.149 | 0.0044 | 0.156 | 0.001 |
| rs1275684 | 26,202,924 | 120 | NA | | 0.318 | NA | NA | 0.170 | 1.68 | 0.094 |
| Gene-based | | | | | | | | | | |

CH: chromosome;
POS: base pair position from build 37;
RA: risk allele;
nSNPs; number of SNPs tested in gene-based analysis;
RAF: risk allele frequency;
β: estimate from the regression model;
P: P value;
Z: Zscore in meta-analysis weighted by the number of SNPs in a gene;
NA: not applicable.
*Gene-based tests were conducted using all genotyped and well imputed (RSQ≧0.8) SNPs from the candidate genes containing at least one SNP with P < 0.05 in both studies.

TABLE 3
PLXNA4 isoform levels in brain from neuropathologically examined AD cases and controls

| | Control | | Early-Stage | | Late-Stage | | All AD | | Late-Stage AD | | All AD vs. | |
|---|---|---|---|---|---|---|---|---|---|---|---|---|
| | (n = 5) | | AD (n = 5) | | AD (n = 9) | | (n = 14) | | vs. Control | | Control | |
| Isoform | Mean | SE | Mean | SE | Mean | SE | Mean | SE | Mean | SE | Mean | SE |
| TS1 | 0.35 | 0.02 | 0.38 | 0.05 | 0.66 | 0.06 | 0.56 | 0.06 | 4.91 | 6.0 × 10⁻⁴ | 3.52 | 0.0028 |
| TS3 | 0.54 | 0.04 | 0.55 | 0.04 | 0.76 | 0.06 | 0.68 | 0.05 | 2.67 | 0.021 | 1.76 | 0.097 |
| TS1/TS3 | 0.67 | 0.09 | 0.67 | 0.05 | 0.88 | 0.06 | 0.81 | 0.05 | 2.02 | 0.066 | 1.38 | 0.18 |

TS1: isoform 1;
TS3: isoform 3;
SE: standard error;
*T-tests account for unequal variances

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. (canceled)

2.-49. (canceled)

50. A method for (i) inhibiting progression of Alzheimer's disease, (ii) inhibiting or reducing neurofibrillary tangles in the brain, (iii) inhibiting or reducing tau phosphorylation in the brain, or (iv) treating a subject having or at risk for Alzheimer's disease, in a subject in need thereof, the method comprising administering to a subject determined to have one or more of AD risk associated single nucleotide polymorphism (SNP) selected from: (i) SNP1 genotype A/A or A/C (or T/T or T/G in the complement) of SEQ ID NO: 1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 (SEQ ID NO: 10) on SEQ ID NO: 1, wherein the SEQ ID NO: 1 is a portion of genomic nucleic acid sequence of PLXNA4; and (iv) any combinations thereof, a therapeutically effective amount of a TS1 PLXNA4 inhibitory agent.

51. The method of claim 50, wherein the subject is determined to have two or more AD risk associated single nucleotide polymorphism (SNP) selected from: (i) SNP1 genotype A/A or A/C (or T/T or T/G in the complement) of SEQ ID NO: 1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 (SEQ ID NO: 10) on SEQ ID NO: 1, wherein the SEQ ID NO. 1 is a portion of genomic nucleic acid sequence of PLXNA4; and (iv) any combinations thereof.

52. The method of claim 50, wherein the subject is determined to have three AD risk associated single nucleotide polymorphism (SNP) selected from: (i) SNP1 genotype A/A or A/C (or T/T or T/G in the complement) of SEQ ID NO: 1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; and (iii) SNP3 genotype C/C or C/A (or G/G or G/T in the complement) of SEQ ID NO: 1, wherein SNP3 identified by rs11761937 (SEQ ID NO: 10) on SEQ ID NO: 1, wherein the SEQ ID NO. 1 is a portion of genomic nucleic acid sequence of PLXNA4.

53. The method of claim 50, wherein the TS1 PLXNA4 inhibitory agent is selected from the group consisting of small molecules, nucleic acids, nucleic acid analogues, peptides, proteins, antibodies, antigen binding fragments of antibodies, and any combinations thereof.

54. The method of claim 50, wherein the TS1 PLXNA4 inhibitory agent is an oligonucleotide.

55. The method of claim 50, wherein the TS1 PLXNA4 inhibitory agent is an anti-miR, antagomir, antisense oligonucleotide, ribozyme, aptamer, siRNA, shRNA, or RNAi agent.

56. The method of claim 50, wherein the TS1 PLXNA4 inhibitory agent does not bind or inhibit TS2 PLXNA4 or TS3 PLXNA4.

57. The method of claim 50, further comprising a step of diagnosing the subject with AD or risk of AD prior to said administering.

58. The method of claim 50, further comprising assaying a biological sample from the subject before onset of said administering, wherein said assaying comprising measuring the absence of presence of a SNP selected from the group consisting of: (i) SNP1 genotype A/A or A/C (or T/T or T/G in the complement) of SEQ ID NO: 1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2 genotype T/T or T/C (or A/A or A/C in the complement) of SEQ ID NO: 1, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; (iii) SNP3 genotype C/C or C/A (or G/G or G/T in the complement) of SEQ ID NO: 1, wherein SNP3 identified by rs11761937 (SEQ ID NO: 10) on SEQ ID NO: 1, wherein the SEQ ID NO. 1 is a portion of genomic nucleic acid sequence of PLXNA4; and (iv) any combinations thereof, wherein presence of one or more of SNP1-SNP3 is indicative of proceeding with said administering regimen.

59. The method of claim 58, wherein said assaying comprises: subjecting the biological sample from a subject to at least one genotyping assay that determines the genotypes of at least one (e.g., one, two, or three) loci, wherein said loci are selected from: (i) SNP1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; (iii) SNP3, wherein SNP3 identified by rs11761937 (SEQ ID NO: 10) on SEQ ID NO: 1, wherein the SEQ ID NO. 1 is a portion of genomic nucleic acid sequence of PLXNA4; and (iv) any combinations thereof.

60. The method of claim 58, wherein said assaying comprises:
a. contacting the biological sample with an allele specific detectable oligonucleotide specific for at least one of the following SNPs: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof;
b. washing the sample to remove unbound oligonucleotide;
c. measuring the intensity of the signal from the bound, detectable bound detectable oligonucleotide;
d. comparing the measured intensity of the signal with a reference value, wherein an increased measured intensity relative to the reference value is indicative of presence of at least one of SNP1-SNP3.

61. The method of claim 50, further comprising assaying a biological sample from the subject before onset of said administering, wherein said assaying comprising measuring the expression or level of TS1 or TS3 PLXNA4, wherein an increased level of expression or amount of TS1 or TS3 PLXNA4, is indicative of proceeding with said administering regimen.

62. The method of claim 61, where said assaying comprises:
a. contacting a biological sample obtained from a subject with a detectable antibody specific for TS1 or TS3 PLXNA4 or detectable nucleic acid for TS1 or TS3 PLXNA4;
b. washing the sample to remove unbound antibody or unbound nucleic acid;
c. measuring the intensity of the signal from the bound, detectable antibody or bound detectable nucleic acid;
d. comparing the measured intensity of the signal with a reference value and if the measured intensity is increased relative to the reference value; and
e. identifying the subject as having an increased probability of having AD.

63. The method of claim 50, wherein the subject in need thereof has Alzheimer's disease.

64. An assay comprising:
a. subjecting a test sample from a subject to at least one genotyping assay that determines the genotypes of at least one (e.g., one, two, or three) loci, wherein said loci are selected from: (i) SNP1, wherein SNP1 is identified by rs277472 (SEQ ID NO: 8) on SEQ ID NO: 1, wherein SEQ ID NO. 1 is a portion of genomic nucleic acid sequence of plexin A4 (PLXNA4); (ii) SNP2, wherein SNP2 is position 132,006,366 of SEQ ID NO: 1 identified by rs10236235 (SEQ ID NO: 9), wherein SEQ ID NO. 1 is a portion of genomic nucleic acid sequence of PLXNA4; (iii) SNP3, wherein SNP3 identified by rs11761937 (SEQ ID NO: 10) on SEQ ID NO: 1, wherein the SEQ ID NO. 1 is a portion of genomic nucleic acid sequence of PLXNA4; and (iv) any combinations thereof; and
b. identifying the subject as having an increased probability of having AD when at least one of the following combinations of SNPs is determined to be present: (i) SNP1 genotype A/A or A/C (or T/T or T/G in the complement) of SEQ ID NO: 1, (ii) SNP2 genotype T/T or T/C (or A/A or A/C in the complement) of SEQ ID NO: 1, (iii) SNP3 genotype C/C or C/A (or G/G or G/T in the complement) of SEQ ID NO: 1, and (iv) any combinations thereof.

65. The assay of claim 64, wherein said loci of step (a) are further selected from: (i) SNP4, wherein SNP4 is identified by rs1593222 (SEQ ID NO: 11) of SEQ ID NO: 1, wherein the SEQ ID NO. 1 is a portion of genomic nucleic acid sequence of PLXNA4; (ii) SNP5, wherein SNP5 is identified by rs6959579 (SEQ ID NO: 12) of SEQ ID NO: 1, wherein the SEQ ID NO. 1 is a portion of genomic nucleic acid sequence of PLXNA4; (iii) SNP6, wherein SNP6 is identified by rs17166339 (SEQ ID NO: 13) of SEQ ID NO: 1, wherein the SEQ ID NO. 1 is a portion of genomic nucleic acid sequence of PLXNA4.

66. The assay of claim 64, further comprising selecting the subject for a treatment regimen, if the subject is identified as having or at risk for Alzheimer's disease.

67. The assay of claim 64, further comprising selecting the subject for administering a TS1 PLXNA4 inhibitory agent, if the subject is identified as having or at risk for Alzheimer's disease.

68. The assay of claim 64, wherein the biological sample is a serum sample.
